(19) United States
(12) Patent Application Publication
(10) Pub. No.: US 2009/0105799 A1
(43) Pub. Date: Apr. 23, 2009

(54) RENAL ASSESSMENT SYSTEMS AND METHODS

(75) Inventors: Neema Hekmat, Mountain View, CA (US); Courtney Yin, Mountain View, CA (US); Frank Altamura, Napa, CA (US); Jeff Elkins, Woodside, CA (US); Aurelio Valencia, Palo Alto, CA (US); Burt Goodson, Fremont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: FlowMedica, Inc., Fremont, CA (US)

(21) Appl. No.: 12/256,154

(22) Filed: Oct. 22, 2008

Related U.S. Application Data
(60) Provisional application No. 60/981,913, filed on Oct. 23, 2007.

(51) Int. Cl. A61F 2/06; A61B 5/02

(52) U.S. Cl. 623/1.11; 600/481; 623/1.42

(57) ABSTRACT

Techniques for assessing a physiological profile of a patient include advancing a catheter shaft of a bifurcated renal catheter system into an aorta of the patient, deploying branches of the bifurcated renal catheter system into the renal arteries of the patient, detecting a renal arterial physiological parameter with a sensing mechanism, and assessing the physiological profile of the patient based on the physiological parameter. Related techniques include modifying or initiating pharmacological or surgical treatments for the patient based on the assessment.

### CROSS REFERENCE TO RELATED APPLICATION

This application is a nonprovisional of, and claims the benefit of the filing date of U.S. Provisional Patent Application No. 60/981,913, entitled “RENAL ASSESSMENT SYSTEMS AND METHODS,” filed Oct. 23, 2007, the entire disclosure of which is incorporated herein by reference for all purposes.

### BACKGROUND OF THE INVENTION

Embodiments of the present invention are generally related to improved devices, systems, and methods for treating or diagnosing a patient. In particular, embodiments encompass techniques for assessing a physiological profile of a patient based on physiological parameters of one or more renal arteries of the patient, and for treating a patient based on such assessments.

Various medical device systems and methods have been previously disclosed for locally delivering fluids or other agents into various body regions, including body lumens such as vessels, or other body spaces such as organs or heart chambers. Local delivery systems may provide for the delivery of drugs or other agents, or may even provide for the delivery of the body's own fluids via shunting or pumping approaches, and the like. Local delivery systems may provide for the introduction of a foreign composition such as a pharmacological agent into the body, which may include a drug or another useful or active agent, and may be in a fluid form or in another form such as a gel, solid, powder, gas, or the like. It is to be understood that reference to only one of the terms fluid, drug, or agent with respect to local delivery descriptions may be made variously in this disclosure for illustrative purposes, but is not generally intended to be exclusive or omissive of the others; they are to be considered interchangeable where appropriate according to one of ordinary skill unless specifically described to be otherwise.

In general, local agent delivery systems and methods are often used for the benefit of achieving relatively high, localized concentrations of agent where injected within the body in order to maximize the intended effects there and while minimizing unintended peripheral effects of the agent elsewhere in the body. Where a particular dose of a locally delivered agent may be efficacious for an intended local effect, the same dose systemically delivered can be substantially diluted throughout the body before reaching the same location. The agent's intended local effect can be equally diluted and efficacy can be compromised. Thus systemic agent delivery often requires higher dosing to achieve an equivalent localized dose for efficacy, often resulting in compromised safety due to for example systemic reactions or side effects of the agent as it is delivered and processed elsewhere throughout the body other than at the intended target.

Exemplary local delivery systems are discussed in, for example, U.S. patent application Ser. No. 11/084,738 filed Mar. 16, 2005; U.S. patent application Ser. No. 11/295,735 filed Dec. 5, 2005; U.S. Pat. No. 7,104,981 issued Sep. 12, 2006; U.S. patent application Ser. No. 11/084,434 filed Mar. 18, 2005; U.S. patent application Ser. No. 11/303,554 filed Dec. 16, 2005; U.S. patent application Ser. No. 11/073,421 filed Mar. 4, 2005; U.S. patent application Ser. No. 11/129,101 filed May 13, 2005; U.S. patent application Ser. No. 11/233,562 filed Sep. 22, 2005; U.S. patent application Ser. No. 11/347,008 filed Feb. 3, 2006; U.S. patent application Ser. No. 11/167,056 filed Jun. 23, 2005; U.S. patent application Ser. No. 11/758,417 filed Jun. 5, 2007; U.S. patent application Ser. No. 11/241,749 filed Sep. 29, 2005; and U.S. patent application Ser. No. 11/548,565 filed Oct. 11, 2006. The entire content of each of these filings is incorporated herein by reference for all purposes.

While these and other proposed systems can be useful in treating conditions such as acute renal failure, and offer benefits for many patients, still further advances would be desirable. In general, it would be desirable to provide improved devices, systems, and methods for treatment, diagnosis, and monitoring of acute renal failure and other conditions of the kidneys or body. It would be particularly desirable if such devices and techniques could increase the overall therapeutic and diagnostic benefit for patients in which they are used, and/or could increase the number of patients who might benefit from renal and other treatments. Ideally, at least some embodiments would include structures and or methods for prophylactic use, potentially altogether avoiding some or all of the deleterious symptoms of acute renal failure.

It would also be desirable to provide techniques for the local delivery of therapies to the renal arteries, in particular when delivered contemporaneous with a diagnostic procedure performed in the patient. The diagnosis or treatment of many different types of medical conditions associated with various different systems, organs, and tissues, may also benefit from the ability to locally deliver fluids or agents in a controlled manner in conjunction with the ability to perform an assessment of physiological parameters in the patient. In particular, various conditions related to the renal system would benefit a great deal from an ability to locally deliver of therapeutic, prophylactic, or diagnostic agents into the renal arteries and also to perform an evaluation of the patient. Embodiments of the present invention provide solutions to at least some of these needs.

### BRIEF SUMMARY OF THE INVENTION

Embodiments of the present invention provide renal catheter systems having bifurcated configurations equipped sensing elements or delivery elements, or combinations of sensing and delivery elements. Exemplary systems and methods involve obtaining real-time evaluation of kidney function, optionally as a function of a targeted renal therapy dosing regimen. These approaches can be used to monitor or assess physiological parameters within a patient, and to determine or modify pharmacological treatments or surgical or other interventions for the patient.

According to embodiments of the present invention, an infusible bifurcated renal catheter system can be used to obtain real-time assessment of renal function and instantaneous feedback or monitoring of any effects of an intervention. For example, an intervention may include a targeted renal therapy or a surgical procedure. An operator or clinician can, based on such assessments of an intervention, implement or make adjustments to a treatment regimen administered to a patient. A treatment regimen could include a pharmacological regimen, a non-pharmacological regimen, or a regimen that includes a pharmacologic and a non-pharmacologic component. For example, a treatment regimen can involve a systematic plan for therapy, prophylaxis, maintenance, and the like. Such implementations or adjustments of a treatment regimen can be determined, at least in part, based on processes performed by a module system associated with the renal catheter system. For example, a clinician, optionally assisted with output from a module system, may implement or make adjustments to a treatment regimen to achieve or pursue desired benefits or effects in the patient. A treatment regimen often involves a systematic plan for therapy, and includes dosing, scheduling, duration, delivery route, and other parameters associated with administration of one or more pharmacological or administered agents, including combinations of such agents. Such regimens can be designed for treatment of an existing disease or condition. A regimen may also be designed to prevent or inhibit the onset of a particular disease, condition, or process that can lead to such a disease or condition. Similarly, regimens can be designed to treat, prevent, or inhibit the recurrence of one or more symptoms of an existing disease or condition, or the recurrence of a process that can lead to or exacerbate such a disease or condition. In some cases, regimens are designed as an attempt to prevent or inhibit the onset or recurrence of such diseases, conditions, or processes. However, it is understood that such attempts may not necessarily result in a cure for the patient or a complete reversal of the disease. In some cases, a patient may not present with a disease or condition, but may present as being exposed or susceptible to, or at risk of developing the disease or condition. Similarly, the patient may present as being potentially exposed or susceptible to, or potentially at risk of developing, the disease or condition. The evaluation and assessment techniques disclosed herein are well suited for use in diagnosing or monitoring a patient who is being treated or who is a candidate for treatment. Assessment or diagnostic evaluations may involve the recognition or detection of a disease or condition, the analysis of physiological or biochemical parameters associated with the cause or effect of a disease or condition, and the like.

In a first aspect, embodiments of the present invention encompass methods of assessing a physiological profile of a patient. An exemplary method includes advancing a catheter shaft of a bifurcated renal catheter system into an aorta of the patient, and deploying a first catheter branch of the bifurcated renal catheter system into a first renal artery of the patient, and a second catheter branch of the bifurcated renal catheter system into a second renal artery of the patient. The method may also include detecting a physiological parameter of the first renal artery, and optionally detecting a physiological parameter of the second renal artery, with a sensing mechanism of the bifurcated renal catheter system. Further, the method may include assessing the physiological profile of the patient based on the physiological parameter of the first renal artery, on the physiological parameter of the second renal artery, or on the physiological parameter of the first renal artery and the physiological parameter of the second renal artery. In some cases, a sensing mechanism is integrated with a catheter shaft, a first catheter branch, a the second catheter branch, or any combination thereof. In some cases, a sensing mechanism is separate from a catheter shaft, a first catheter branch, and a second catheter branch. According to some embodiments, a first catheter branch includes a first branch sensing element, and a second catheter branch includes a second branch sensing element, and a method involves detecting a physiological parameter of a first renal artery with a first branch sensing element, and optionally detecting a physiological parameter of a second renal artery with a second branch sensing element.

In some aspects, a method may include advancing a catheter shaft of a bifurcated renal catheter system into an inferior vena cava of the patient. Relatedly, a method may include deploying a first catheter branch of a bifurcated renal catheter system into a first renal vein of the patient, and a second catheter branch of a bifurcated renal catheter system into a second renal vein of the patient. Further, a method may include detecting a physiological parameter of a first renal vein, and optionally detecting a physiological parameter of a second renal vein, with a sensing mechanism of a bifurcated renal catheter system. A method may also include assessing the physiological profile of the patient based on a physiological parameter of a first renal vein, on the physiological parameter of a second renal vein, or on a physiological parameter of a first renal vein and a physiological parameter of a second renal vein.

In some aspects, a method may include delivering a first amount of a first pharmacological agent to a first renal artery, and optionally delivering a second amount of a second pharmacological agent to a second renal artery, with an agent delivery mechanism of a bifurcated renal catheter system. A related method may include detecting a subsequent physiological parameter of the first renal artery, and optionally detecting a subsequent physiological parameter of the second renal artery, with a sensing mechanism of the bifurcated renal catheter system. A related method may also include assessing an effect of the first amount of the first pharmacological agent on the physiological profile of the patient based on the subsequent physiological parameter of the first renal artery, and optionally assessing the effect of the of the second amount of the second pharmacological agent on the physiological profile of the patient based on the subsequent physiological parameter of the second renal artery. A pharmacological agent or material may include a contrast solution, a chemotherapy agent, an antioxidant, sodium bicarbonate, acetylcysteine, a chelation agent, an anti-inflammatory agent, fenoldopam mesylate, a vasodilator, prostaglandin, a diuretic, a loop diuretic, furosemide, an antibiotic agent, a bactericidal agent, a bacteriostatic agent, a neurohormonally active agent, a natriuretic peptide, A-type natriuretic peptide, B-type natriuretic peptide, C-type natriuretic peptide, a synthetic natriuretic peptide, a bio-engineered natriuretic peptide, or the like. In related aspects, a method may include determining a third amount of a third pharmacological agent based on the effect of the first amount of the first pharmacological agent, and optionally based on the effect of the second amount of the second pharmacological agent, and delivering the third amount of the third pharmacological agent to the first renal artery, to the second renal artery, or to both, with the agent delivery mechanism of the bifurcated renal catheter.

In some aspects, a method may include performing a surgical procedure on the patient, detecting a subsequent physiological parameter of the first renal artery, and optionally detecting a subsequent physiological parameter of the second renal artery, with a sensing mechanism of the bifurcated renal catheter system, and assessing an effect of the surgical procedure on the physiological profile of the patient based on the subsequent physiological parameter of the first renal artery, and optionally assessing the effect of the surgical procedure on the physiological profile of the patient based on the subsequent physiological parameter of the second renal artery. An exemplary surgical procedure may involve or include a stenting procedure, a bypass procedure, an angiographic procedure, a percutaneous coronary intervention, an invasive surgical procedure, or the like. An exemplary physiological parameter of a blood vessel, for example a renal artery, may include a blood concentration or presence of a physiological marker such as aldosterone, renin, angiotensin II, serum creatinine (SrCr), urea, neutrophil gelatinase-associated lipocalin (NGAL), cystanin C, acetylcholine, bradykinin, blood urea nitrogen (BUN), calcium, potassium, sodium, chloride, bicarbonate, oxygen, nitric oxide (NO), nitric oxide synthase (NOS), reactive oxygen species (ROS), iron, an iron-based biochemical derivative such as serum ferritin, blood pH, and the like. In some cases, a physiological parameter of a blood vessel may include a blood concentration or presence of an inflammatory marker such as a polymorphonuclear leukocyte (PMN), an interleukin-8 (IL-8), IL-13, IL-17, or the like. In some cases, a physiological parameter of a blood vessel may include a blood concentration or presence of a blood chemotaxis indicator such as a chemotaxis protein (MCP), methylesterase, methyltransferase, and the like. In some cases, a physiological parameter of a blood vessel may include a blood concentration of a contrast solution. In some cases, a physiological parameter of a blood vessel may include a physical marker such as a renal artery blood flow velocity, a volumetric blood flow rate, a total renal blood flow, an inner arterial wall shear stress, a pressure, a luminal diameter, a stenosis measure, a clot measure, a particle measure, a temperature, and the like.

According to some embodiments, a method may include detecting a physiological parameter at a third location within the patient with a sensing mechanism of the bifurcated renal catheter system, and assessing the physiological profile of the patient based on the physiological parameter of the third location. The third location may include a location within an aorta of the patient. In some cases, the third location may include a location within a systemic vessel of the patient. An exemplary sensing mechanism may include an ultrasonic transducer sensor, an expandable and retractable frame, a flow guided sensor, a balloon, a mesh umbrella, a flow meter, a shear stress sensor, a pressure sensor, a temperature sensor, a flow velocity sensor, a volumetric flow sensor, a Doppler sensor, a biochemical sensor, or the like.

In another aspect, embodiments of the present invention encompass a bifurcated renal catheter system for assessing a physiological profile of a patient. The system can include, for example, a catheter having a shaft coupled with a first catheter branch and a second catheter branch. The system may also include a sensing mechanism. In some cases, a system can include an assessment module. In some cases, a sensing mechanism includes a first sensor coupled with a first catheter branch, and a second sensor coupled with a second catheter branch. In some cases, a sensing mechanism includes a sensor coupled with a catheter shaft. A sensing mechanism may include an ultrasonic transducer sensor, an expandable and retractable frame, a flow guided sensor, a balloon, a mesh umbrella, a flow meter, a shear stress sensor, a pressure sensor, a temperature sensor, a flow velocity sensor, a volumetric flow sensor, a Doppler sensor, a biochemical sensor, and the like. In some cases, a system may include a monitoring system which can communicate with or receive information, data, or signals from the sensing mechanism. According to some embodiments, a first catheter branch includes a first infusion port, and a second catheter branch includes a second infusion port. A guide sheath can be configured to receive the catheter shaft, a system monitor coupled with the sensing mechanism, and an infusion pump coupled with the first and second infusion ports. In some cases, a sensing mechanism includes an expandable and retractable frame coupled with a control wire. Optionally, the frame in a first configuration can be expanded radially from the first catheter branch when the control wire is advanced in a distal direction relative to the first catheter branch, and the frame in a second configuration can be retracted radially toward the first catheter branch when the control wire is withdrawn in a proximal direction relative to the first catheter branch. A sensing mechanism may include a flow rate sensor coupled with a distal portion of the first catheter branch. Optionally, a flow rate sensor may be coupled with the distal portion via a tether. According to some embodiments, a sensing mechanism may include an expandable and retractable frame coupled with the first catheter branch, and a flow rate sensor coupled with the first catheter branch. In some cases, a sensing mechanism may include a multi-prong balloon. In some cases, a sensing mechanism may include a force transducer coupled with the first catheter branch, and a drag mechanism coupled with the force transducer. Optionally, a sensing mechanism may include an expandable and retractable member coupled with the first catheter branch, and a shear stress sensor coupled with the expandable and retractable member. In some cases, a sensing mechanism includes a stent releasably attached with the first catheter branch, and a shear stress sensor coupled with the stent. In some cases, a sensing mechanism includes a first pressure sensor coupled with the first catheter branch, a second pressure sensor coupled with the second catheter branch, and a third pressure sensor coupled with the catheter shaft. In some cases, a sensing mechanism includes a temperature sensor coupled with a distal portion of the first catheter branch, and an injection port disposed at a proximal portion of the first catheter branch. In some cases, a sensing mechanism includes a stent releasably attached with the first catheter branch, and a distal pressure sensor and a proximal pressure sensor coupled with the stent. In some cases, a sensing mechanism includes a sensing element coupled with a deformable wire, and the deformable wire is disposed at least partially within the catheter shaft and the first catheter branch.

In another aspect according to embodiments of the present invention, a method of determining a physiological profile of a patient includes receiving a physiological parameter of a first renal artery, and optionally receiving a physiological parameter of the second renal artery, at an input module of a monitor and control system, where the input module includes a tangible medium embodying machine-readable code. The method may also include determining the physiological profile of the patient with an assessment module of the monitor and control system, where the assessment module includes a tangible medium embodying machine-readable code. In some cases, a method includes transmitting the physiological profile of the patient to a visual output device, an auditory output device, a printer device, a processor device, a memory device, a data transmission device, or the like. In some cases, a method may include determining a patient treatment, based on the physiological profile, with a treatment module of the monitor and control system, where the treatment module includes a tangible medium embodying machine-readable code. According to some embodiments, the process of determining the patient treatment can include calculating an amount of a treatment agent to be delivered to the first renal artery of the patient. According to some embodiments, the process of determining the patient treatment can include determining a treatment agent to be delivered to the first renal artery of the patient. In some embodiments, a method may include advancing a catheter shaft of a bifurcated renal catheter system into an aorta of the patient, deploying a first catheter branch of the bifurcated renal catheter system into the first renal artery of the patient, and deploying a second catheter branch of the bifurcated renal catheter system into the second renal artery of the patient. A method may also include detecting the physiological parameter of the first renal artery, and optionally detecting the physiological parameter of the second renal artery, with a sensing mechanism of the bifurcated renal catheter system. Some methods may include the step of administering a treatment to the patient, and determining a subsequent physiological profile of the patient after or while administering the treatment the patient. Some methods may include determining a subsequent treatment for the patient, based on the subsequent physiological profile. It is appreciated that in many cases, method steps may be performed by a computer or by a human.

In another aspect, embodiments of the present invention encompass a bifurcated renal catheter system for assessing a physiological profile of a patient. The system may include, for example, a catheter having a shaft coupled with a first catheter branch and a second catheter branch, and a sensing mechanism having a first sensor coupled with the first catheter branch, and optionally a second sensor coupled with the second catheter branch. The catheter system may also include a monitor and control system with an input module having a tangible medium embodying machine-readable code configured to receive an input from the sensing mechanism, and an assessment module having a tangible medium embodying machine readable code configured to assess the physiological profile of the patient based on the input.

In a further aspect, embodiments of the present invention encompass a module system for determining a treatment for a patient. The system may include, among other things, a catheter having a shaft coupled with a first catheter branch and a second catheter branch, and a sensing mechanism having a first sensor coupled with the first catheter branch, and optionally a second sensor coupled with the second catheter branch. The module system also includes a monitor and control system with an input module having a tangible medium embodying machine-readable code configured to receive an input from the sensing mechanism, an assessment module having a tangible medium embodying machine readable code configured to perform an assessment of the physiological profile of the patient based on the input, and a treatment module having a tangible medium embodying machine-readable code configured to determine a patient treatment based on the assessment.

For a fuller understanding of the nature and advantages of the present invention, reference should be had to the ensuing detailed description taken in conjunction with the accompanying drawings.

### BRIEF DESCRIPTION OF THE DRAWINGS

FIG. 1 shows a catheter system according to embodiments of the present invention.

FIG. 2A illustrates a catheter system according to embodiments of the present invention.

FIG. 2B illustrates a catheter system according to embodiments of the present invention.

FIG. 3A illustrates a catheter system according to embodiments of the present invention.

FIG. 3B illustrates a catheter system according to embodiments of the present invention.

FIGS. 4A to 4C depict aspects of a catheter system according to embodiments of the present invention.

FIGS. 5A to 5E depict aspects of catheter systems according to embodiments of the present invention.

FIG. 6 shows aspects of a catheter system according to embodiments of the present invention.

FIG. 7 shows aspects of a catheter system according to embodiments of the present invention.

FIG. 8 illustrates aspects of a catheter system according to embodiments of the present invention.

FIG. 9 illustrates aspects of a catheter system according to embodiments of the present invention.

FIG. 10 illustrates aspects of a catheter system according to embodiments of the present invention.

FIG. 11 illustrates aspects of a catheter system according to embodiments of the present invention.

FIG. 11A illustrates aspects of a catheter system according to embodiments of the present invention.

FIGS. 12A and 12B show aspects of a catheter system according to embodiments of the present invention.

FIG. 13 illustrates aspects of a module system according to embodiments of the present invention.

FIGS. 14A to 14C show physiological parameters associated with renal function as a function of a targeted renal therapy dosage, according to embodiments of the present invention.

### DETAILED DESCRIPTION OF THE INVENTION

Embodiments of the present invention encompass systems and methods for the real-time assessment of renal function and other related physiological parameters relevant to the renal arteries via a bifurcated catheter platform and monitoring system. In some embodiments, a sensing-capable bifurcated catheter platform can be embodied in a configuration that includes a bilateral renal artery access cannulation apparatus. Such a configuration can provide for the real-time assessment of renal function or those physiological parameters measurable via the renal arteries, as an adjunct to a surgical intervention or medical procedure or in situations where monitoring of such parameters is desired. In some embodiments, a sensing-capable bifurcated catheter platform can be embodied in a configuration that includes a bilateral renal artery access cannulation apparatus providing for the infusion of solutions or other materials of choice directly to the renal arteries in addition to its sensing capabilities. An infusion catheter apparatus may provide a real-time assessment of the effects of an infusing solution on physiological parameters of interest such as those measurable via the renal arteries. This assessment may be performed during or in conjunction with a targeted renal therapy treatment. Exemplary embodiments allow for real-time monitoring and evaluation of the efficacy and safety of targeted renal therapy administration in light of such physiological parameters. This real-time monitoring of physiological parameters provides an end-user the ability to make adjustments in dosage and/or drug as necessary or desired. In addition, exemplary embodiments permit real-time assessment and control of renal function or related physiological parameters during times when infusion through the catheter may or may not occur.

In some embodiments, sensing elements are embedded within a catheter, and may be located on one or more branches of a branched catheter. In cases where simultaneous and independent measurement of function of both kidneys may be desired, sensing elements may be located on both branches of a bifurcated catheter, for example. Often, catheter system configurations as described herein are intended for placement within the renal arteries. In instances where measurements may be desired from the venous circulation for differential measurements between arterial and venous circulation, a second bifurcated catheter may be placed within the renal veins. Such techniques can be helpful in evaluating the level of excretion of a certain compound, molecule, or ion by the kidneys from circulation or other parameters relevant to relative renal function. Examples of cases where such a differential measurement may be beneficial are detailed herein.

Any of a variety of physiological markers may be measured via sensing elements, which may be embedded within or otherwise associated with a catheter branch or shaft of a bifurcated catheter system. These physiological markers for real-time assessment may include: aldosterone, renin, angiotensin II, serum creatinine (SrCr), urea, NGAL (Neutrophil gelatinase-associated lipocalin), cystanin C, acetylcholine, bradykinin, pK, pH, BUN (blood urea nitrogen), electrolytes (e.g. calcium, potassium, sodium), oxygen (such as via a pulse oximetry-based sensor), nitric oxide, chloride, bicarbonate, nitric oxide synthase (NOS), reactive oxygen species (ROS), iron, an iron-based biochemical derivative, blood pH, or the like. A physiological parameter may also include a blood concentration of a contrast solution. For such physiological marker assessments, fiberoptic or micro spectroscopy may be implemented in the bifurcated catheter system. Similarly, a bifurcated catheter system can include nanotechnology or pharmacological assays. In addition sensors specific for inflammatory markers, such as polymorphonuclear leukocyte (PMN), an interleukin-8 (IL-8), IL-13, IL-17, and the like, and chemotaxis, such as chemotaxis protein (MCP), methylesterase, methyltransferase, and the like, may be used for assessing renal function. Other physical parameters that may be assessed via a bifurcated catheter system include renal artery blood flow velocity, volumetric blood flow rate, pressure, luminal diameter, temperature, total renal blood flow, inner arterial wall shear stress, stenosis measurement, clot measurement, particle measurement, and the like.

Any of a variety of pharmacologic or other agents can be administered to a patient via a bifurcated catheter. Exemplary agents include, without limitation, a contrast solution, a chemotherapy agent, an antioxidant, sodium bicarbonate, acetylcysteine, a chelation agent, an anti-inflammatory agent, fenoldopam mesylate, a vasodilator, prostaglandin, a diuretic, a loop diuretic, furosemide, an antibiotic agent, a bactericidal agent, a bacteriostatic agent, a neurohormonally active agent, a natriuretic peptide, A-type natriuretic peptide, B-type natriuretic peptide, C-type natriuretic peptide, a synthetic natriuretic peptide, a bio-engineered natriuretic peptide, or the like. Embodiments of the present invention may encompass any of a variety of surgical procedures, including without limitation, a stenting procedure, a bypass procedure, an angiographic procedure, a percutaneous coronary intervention, an invasive surgical procedure, or the like.

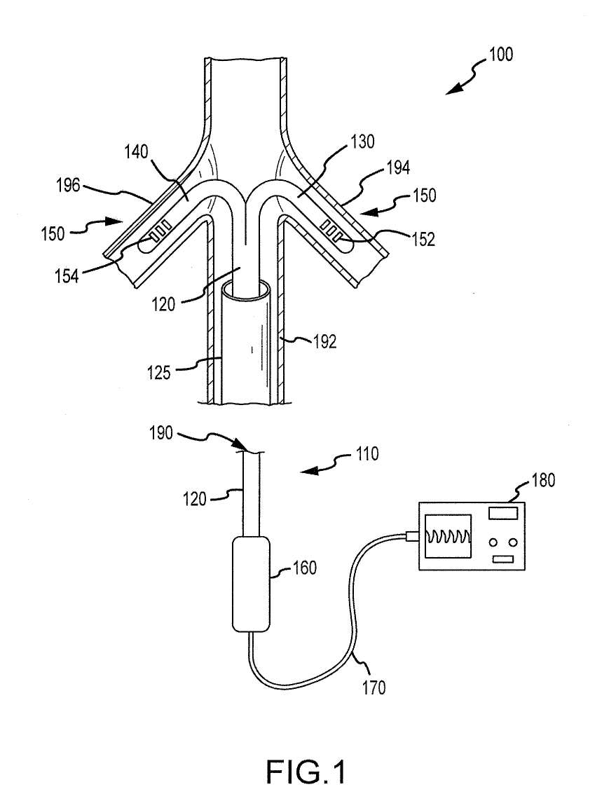

Turning now to the drawings, FIG. 1 shows a bifurcated renal catheter system 100 for assessing a physiological profile of a patient, according to embodiments of the present invention. Bifurcated renal catheter system 100 includes a catheter 110 having a shaft 120 coupled with a first catheter branch 130 and a second catheter branch 140. Bifurcated renal catheter system 100 also includes a sensing mechanism 150 having one or more sensing members 152 coupled with the first catheter branch 130 and one or more second sensing members 154 coupled with the second catheter branch 140. Catheter shaft 120 is coupled with a catheter hub 160. As depicted here, system 100 also includes a guide sheath 125 that is configured to receive catheter shaft 120. A sensor data cable 170 can transmit signals or data from sensing mechanism 150 to a module system 180. An operator can use system 100 to assess a physiological profile of a patient. An exemplary method may involve inserting guide sheath 125 through a minimally invasive incision 190 in a patient, and into or toward a descending aorta, such as a thoracic aorta or abdominal aorta 192 of the patient. The operator may advance catheter shaft 120 through guide sheath 125, and toward first and second renal arteries 194, 196. The operator can deploy first catheter branch 130 of bifurcated renal catheter system 100 into first renal artery 194. The operator can also deploy second catheter branch 140 of bifurcated renal catheter system 100 into second renal artery 196. Sensing mechanism 150 can be used to detect one or more physiological parameters within the patient. For example, first sensing member 152 can be used to detect a physiological parameter of first renal artery 194. Similarly, second sensing member 154 can be used to detect a physiological parameter of second renal artery 196. It is then possible to assess a physiological profile of the patient based on the physiological parameter of first renal artery 194, on the physiological parameter of second renal artery 196, or based on both the physiological parameter of first renal artery 194 and the physiological parameter of second renal artery 196.

Sensing mechanism 150 can include any of a variety of sensing members, including without limitation ultrasonic transducer sensors, expandable and retractable frames, flow guided sensors, balloons, mesh umbrellas, flow meters, shear stress sensors, pressure sensors, temperature sensors, flow velocity sensors, biochemical sensors, volumetric flow sensors, Doppler-based sensors, and the like. An operator can thus use bifurcated renal catheter system 100 for the real-time assessment of renal function and other related physiological parameters relevant to the renal arteries. This assessment can be performed as an adjunct to a surgical intervention or medical procedure, or in situations where monitoring of such parameters is desired. According to some embodiments, a sensing mechanism may be integrated with or separate from a catheter shaft, a first catheter branch, or a second catheter branch.

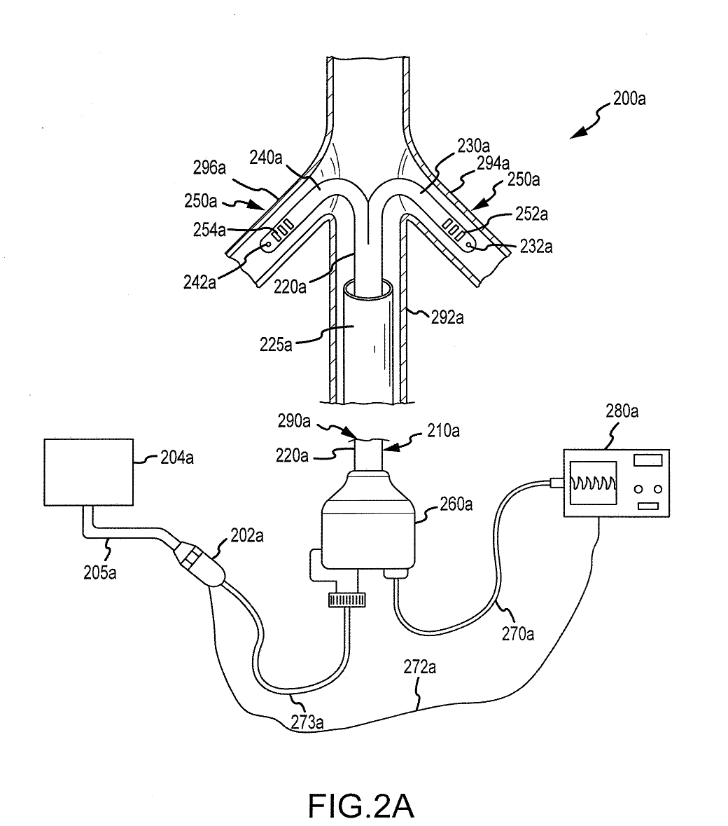

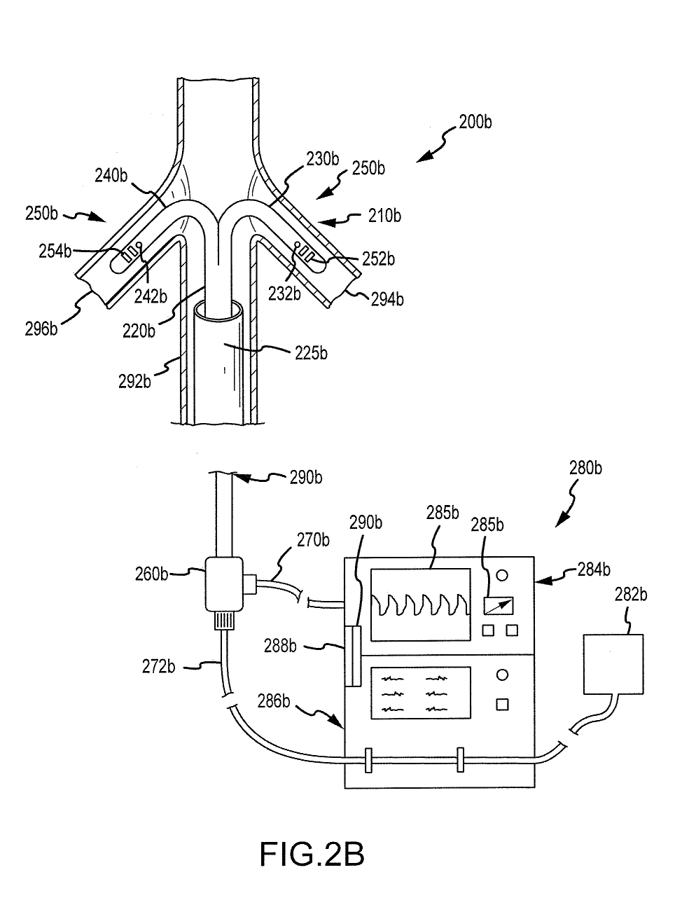

FIG. 2A shows a bifurcated renal catheter system 200a for assessing a physiological profile of a patient, according to embodiments of the present invention. Bifurcated renal catheter system 200a includes a catheter 210a having a shaft 220a coupled with a first catheter branch 230a and a second catheter branch 240a. Bifurcated renal catheter system 200a also includes a sensing mechanism 250a having one or more sensing members 252a coupled with the first catheter branch 230a and one or more second sensing members 254a coupled with the second catheter branch 240a. Catheter shaft 220a is coupled with a catheter hub 260a. As depicted here, system 200a also includes a guide sheath 225a that is configured to receive catheter shaft 220a. A data cable 270a can transmit signals or data between sensing mechanism 250a and a module system 280a. An operator can use system 200a to assess a physiological profile of a patient. An exemplary method may involve inserting guide sheath 225a through a minimally invasive incision 290a in a patient, and into or toward an aorta or abdominal aorta 292a of the patient. The operator may advance catheter shaft 220a through guide sheath 225a, and toward first and second renal arteries 294a, 296a. The operator can deploy first catheter branch 230a of bifurcated renal catheter system 200a into first renal artery 294a. The operator can also deploy second catheter branch 240a of bifurcated renal catheter system 200a into second renal artery 296a. Sensing mechanism 250a can be used to detect one or more physiological parameters within the patient. For example, first sensing member 252a can be used to detect a physiological parameter of first renal artery 294a. Similarly, second sensing member 254a can be used to detect a physiological parameter of second renal artery 296a. It is then possible to assess a physiological profile of the patient based on the physiological parameter of first renal artery 294a, on the physiological parameter of second renal artery 296a, or based on both the physiological parameter of first renal artery 294a and the physiological parameter of second renal artery 296a.

The catheter branches may include one or more infusion ports. For example, as depicted here, first catheter branch 230a also includes a first infusion port 232a, and second catheter branch 240a includes a second infusion port 242. A data cable 272a can transmit signals or data between module system 280a and an infusion pump 202a. An infusion tube 273a can act as a conduit for infusate between infusion pump 202a and the infusion ports. In some embodiments, infusion pump 202a can include or be coupled with a source of fluid or other agent. For example, infusion pump 202a can be coupled with an intravenous bag 204a via an intravenous tubing 205a. An operator can use system 200a to deliver a fluid, agent, or other material to the patient, via infusion pump 202a and at least one of the first infusion port 232a and the second infusion port 242a. Often, the amount or type of material administered to the patient is based on the physiological profile of the patient, as assessed via module system 280a.

Use of bifurcated renal catheter system 200a allows an operator to obtain a real-time assessment of renal function and other related physiological parameters relevant to the renal arteries. Relatedly, an operator can use bifurcated renal catheter system 200 to deliver solutions and other materials directly to the renal arteries in addition to sensing physiological parameters within the renal arteries. Catheter system 200a can provide an operator or user with a real-time assessment or evaluation of any effects of an infusing solution, which may be given as part of a targeted renal therapy treatment. Hence, the operator can use system 200a to evaluate physiological parameters of interest, which may or may not change in response to targeted renal therapy or other surgical or medical interventions that are performed on the patient. In this way, the operator can enjoy real-time monitoring and evaluation of the efficacy and safety of targeted renal therapy, or other interventions, in light of such physiological parameters. Real-time monitoring of physiological parameters allows the user or operator to make adjustments in drugs or dosages to achieve or pursue desired pharmacological benefits. An operator can also use system 200a to obtain real-time assessment and control or modulation of renal function or related physiological parameters, during times when infusion through the catheter may not occur. It is appreciated that sensing members may be embedded within the catheter, and may be located on one or more branches of the catheter. In cases where simultaneous and independent measurement of function of both kidneys may be desired, sensing elements may be located on both catheter branches. Hence, an operator can use bifurcated renal catheter system 200a, which may include a monitoring or control system, to display and provide feedback of physiological parameters of interest.

FIG. 2B shows a bifurcated renal catheter system 200b for assessing a physiological profile of a patient, according to embodiments of the present invention. Bifurcated renal catheter system 200b includes a catheter 210b having a shaft 220b coupled with a first catheter branch 230b and a second catheter branch 240b. Bifurcated renal catheter system 200b also includes a sensing mechanism 250b having one or more sensing members 252b coupled with the first catheter branch 230b and one or more second sensing members 254b coupled with the second catheter branch 240b. Catheter shaft 220b is coupled with a catheter hub 260b. As depicted here, system 200b also includes a guide sheath 225b that is configured to receive catheter shaft 220b. A data cable 270b can transmit signals or data between sensing mechanism 250b and a module system 280b. An infusion tube 272b can be used to deliver an infusate or solution to a first infusion port 232b disposed on first catheter branch 230b, and optionally to a second infusion port 242b disposed on second catheter branch 240b. An operator can use system 200b to assess a physiological profile of a patient. An exemplary method may involve inserting guide sheath 225b through a minimally invasive incision 290b in a patient, and into or toward an aorta or abdominal aorta 292b of the patient. The operator may advance catheter shaft 220b through guide sheath 225b, and toward first and second renal arteries 294b, 296b. The operator can deploy first catheter branch 230b of bifurcated renal catheter system 200b into first renal artery 294b. The operator can also deploy second catheter branch 240b of bifurcated renal catheter system 200b into second renal artery 296b. Sensing mechanism 250b can be used to detect one or more physiological parameters within the patient. For example, first sensing member 252b can be used to detect a physiological parameter of first renal artery 294b. Similarly, second sensing member 254b can be used to detect a physiological parameter of second renal artery 296b. It is then possible to assess a physiological profile of the patient based on the physiological parameter of first renal artery 294b, on the physiological parameter of second renal artery 296b, or based on both the physiological parameter of first renal artery 294b and the physiological parameter of second renal artery 296b.

The catheter branches may include one or more infusion ports. For example, as depicted here, first catheter branch 230b also includes a first infusion port 232b, and second catheter branch 240b includes a second infusion port 242b. Infusate can be delivered from a source 282b of fluid or other agent, to one or more infusion ports. Source 282b can include, for example, one or more intravenous bags. As shown here, module system 280b includes a monitor module 284b and a delivery module 286b. Module system 280b can, for example, receive a physiological parameter of first renal artery 294b, and optionally receive a physiological parameter of second renal artery 269b, at monitor module 284b. In some embodiments, module system 280b includes an assessment module 288b, which can be used to determine a physiological profile of the patient based on the physiological parameter of first renal artery 294b, optionally in conjunction with the physiological parameter of second renal artery 269b. Monitor module 284b can include one or more output devices 285b, such as a visual output device or display, an auditory output device, a printer device, a processor device, a memory device, a data transmission device, or the like. Module system 280b can also include a treatment module 290b that is configured to determine a patient treatment based on a physiological parameter of a renal artery, or based on an assessment of a physiological parameter of a renal artery. In some cases, treatment module 290b can determine a patient treatment based on a physiological profile of the patient. A patient treatment may include an amount of a treatment or diagnostic agent to be delivered to a renal artery of the patient. Module system 280b can be used to implement a treatment via delivery module 286b. For example, delivery module 286b may include an infusion pump that can facilitate and control the delivery of an infusate from source 282b to an infusion port 232b, 242b. The amount, type, and timing of the infusate that is administered can be controlled by delivery module 286b, for example based on a patient treatment that is determined by treatment module 290b.

Use of bifurcated renal catheter system 200b allows an operator to obtain a real-time assessment of renal function and other related physiological parameters relevant to the renal arteries. Relatedly, an operator can use bifurcated renal catheter system 200b to deliver solutions and other materials directly to the renal arteries in addition to sensing physiological parameters within the renal arteries. Catheter system 200b can provide an operator or user with a real-time assessment or evaluation of any effects of an infusing solution, which may be given as part of a targeted renal therapy treatment. Hence, the operator can use system 200b to evaluate physiological parameters of interest, which may or may not change in response to targeted renal therapy or other surgical or medical interventions that are performed on the patient. In this way, the operator can enjoy real-time monitoring and evaluation of the efficacy and safety of targeted renal therapy, or other interventions, in light of such physiological parameters. Real-time monitoring of physiological parameters allows the user or operator to make adjustments in drugs or dosages to achieve or pursue desired pharmacological benefits. An operator can also use system 200b to obtain real-time assessment and control or modulation of renal function or related physiological parameters, during times when infusion through the catheter may not occur. It is appreciated that sensing members may be embedded within the catheter, and may be located on one or more branches of the catheter. In cases where simultaneous and independent measurement of function of both kidneys may be desired, sensing elements may be located on both catheter branches. Hence, an operator can use bifurcated renal catheter system 200b, which may include a monitoring or control system, to display and provide feedback of physiological parameters of interest.

Hence, monitor module 284b can provide an operator or clinician with feedback of real-time measurements made via catheter 210b. Monitor module 284b can include an external or internal signal processing unit and display screen, for example. Assessment module 288b can include data analysis programs that allow the operator or clinician to monitor and evaluate any changes in measurements from baseline. Such measurements can be compared to an absolute threshold value which can indicate a critical or alert level. In some embodiments, such data analyses programs may carry an alert system to provide the operator or clinician with a visual indication or audible alarm if a certain physiological parameter has reached a critical limit or threshold in terms of absolute measurement or magnitude change from baseline. In the case where catheter system 200b includes an infusion capability, module system 280b may allow the user to assess the efficacy of a targeted renal therapy and modulate the administered drug dosage or drug infusion rate as deemed appropriate to optimize or otherwise modulate its effects.

In some embodiments of the present invention, module system 280b can be used to automate the modulation of the administered drug dosage, such that an optimal or desired effect of a targeted renal therapy is achieved. Module system 280b can integrate an infusion pump apparatus or module 286b, where an infusion line 272b is connected to an infusible configuration of catheter system 210b. As the sensing elements within the catheter provide the user with feedback regarding physiological parameters of interest, a control system within module system 280b can utilize these input signals as negative feedback to the control a processor to modulate the infusion rate of the pump. Thus, where a targeted renal therapy is administered via an infusible configuration of the catheter system, such an integrated negative feedback control system allows for an automated modulation in drug dose to achieve an optimal or desired effect of the targeted renal therapy.

In some embodiments, an infusible catheter can present a double lumen configuration, whereby separate infusion lumens are disposed within the catheter shaft, and optionally within infusion tube 272b. Thus, separate infusion lumens can provide for independent delivery of infusate to separate infusions ports. For example, a first infusate can be delivered to first infusion port 232b, and a second infusate can be delivered to second infusion port 242b. In some cases, the same infusate is administered through two infusion ports, albeit at different rates or different amounts, or otherwise according to different dosing schedules. Independent administration protocols such as these may be based on independent sensing techniques. For example, an administration protocol configured for delivery through first infusion port 232b can be based on physiological parameter data received from first sensing member 252b, and an administration protocol configured for delivery through second infusion port 242b can be based on physiological parameter data received from first sensing member 254b. These procedures can involve independent manual or automatic control of infusate delivery, facilitated by module system 280b. Accordingly, it is possible to modulate or control the effects of a targeted renal therapy on each of the renal arteries and corresponding kidneys via independent infusion control techniques, based on independent sensing protocols.

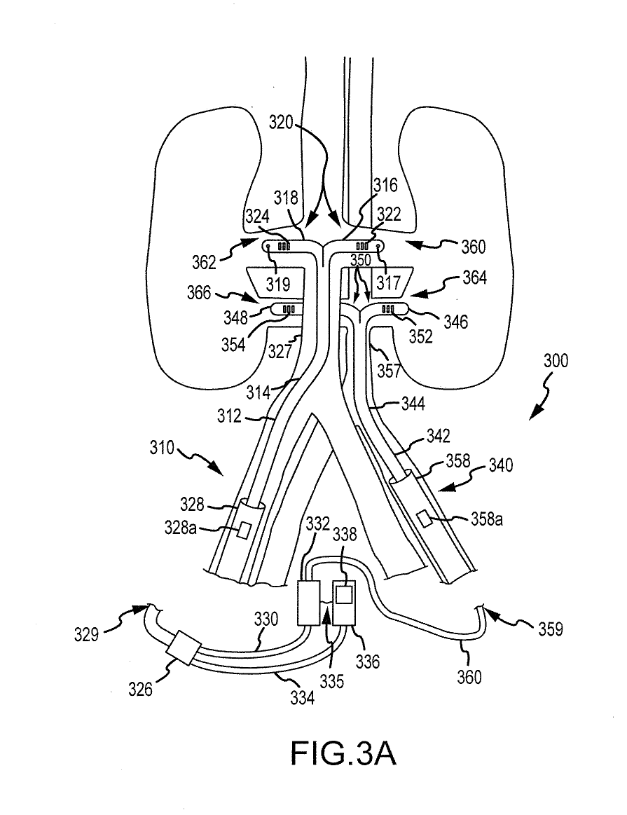

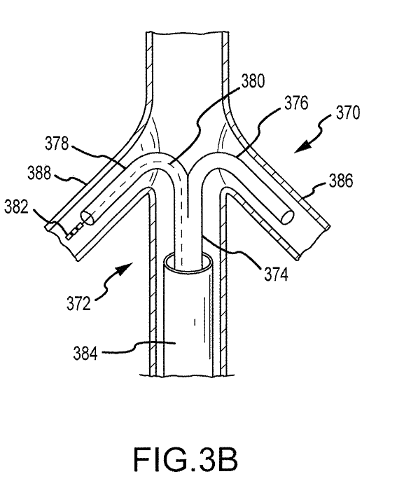

As shown in FIG. 3A, an operator may deploy a second catheter into the venous system of a patient, in addition to deploying a first catheter in the arterial system. FIG. 3A illustrates a renal catheter system 300 for assessing a physiological profile of a patient, according to embodiments of the present invention. Renal catheter system 300 includes a first or arterial bifurcated renal catheter system 310, which in turn includes a catheter 312 with a shaft 314. First catheter system 310 also includes a first catheter branch 316 and a second catheter branch 318, each coupled with catheter shaft 314. Further, first bifurcated renal catheter system 310 includes a sensing mechanism 320 having one or more sensing members 322 coupled with the first catheter branch 316 and one or more second sensing members 324 coupled with the second catheter branch 318. Catheter shaft 312 is coupled with a catheter hub 326. As depicted here, first catheter system 310 may also include a guide sheath 328 that is configured to receive catheter shaft 312. Guide sheath 328 may include a sensing member 328a, which can sense or detect conditions within the aorta, for example. A data cable 330 can transmit signals or data between sensing mechanism 320 and a module system 332. Catheter branches 316, 318 may also include one or more infusion ports. For example, as depicted here, first catheter branch 316 includes a first infusion port 317, and second catheter branch 318 includes a second infusion port 319. A data cable 335 can transmit signals or data between module system 332 and an infusion pump 336. An infusate tube 334 can provide a passage for fluid between pump 336 and the infusion ports. In some embodiments, infusion pump 336 can include or be coupled with a source of fluid or other agent. For example, infusion pump 336 can include an agent source 338. An operator can use first catheter system 310 to deliver a fluid, agent, or other material to the patient, via infusion pump 336 and at least one of the first infusion port 317 and the second infusion port 319.

Renal catheter system 300 also includes a second or venous bifurcated renal catheter system 340, which in turn includes a catheter 342 with a shaft 344. Second catheter system 340 also includes a first catheter branch 346 and a second catheter branch 348, each coupled with catheter shaft 344. Further, first bifurcated renal catheter system 340 includes a sensing mechanism 350 having one or more sensing members 352 coupled with the first catheter branch 346 and one or more second sensing members 354 coupled with the second catheter branch 348. As depicted here, second catheter system 340 may also include a guide sheath 358 that is configured to receive catheter shaft 342. A data cable 360 can transmit signals or data between sensing mechanism 350 and module system 332.

An operator can use system 300 to assess a physiological profile of a patient. An exemplary method may involve inserting guide sheath 328 through a minimally invasive incision 329 and into or toward a descending aorta, such as a thoracic aorta or abdominal aorta 327 of the patient, and inserting guide sheath 358 through a minimally invasive incision 359 and into or toward an inferior vena cava 357. Guide sheath 358 may include a sensing member 358a, which can sense or detect conditions within the inferior vena cava, for example. Minimally invasive incision 329 may provide access to, for example, a femoral or iliac artery of the patient. Similarly, minimally invasive incision 359 may provide access to, for example, a femoral or iliac vein of the patient. The operator may advance catheter shaft 314 through guide sheath 328, and toward first and second renal arteries 360, 362, and may advance catheter shaft 344 through guide sheath 358, and toward first and second renal veins 364, 366. The operator can deploy first catheter branch 316 of bifurcated renal catheter system 310 into first renal artery 360, and second catheter branch 318 of bifurcated renal catheter system 310 into second renal artery 362. The operator can deploy first catheter branch 346 of bifurcated renal catheter system 340 into first renal vein 364, and second catheter branch 348 of bifurcated renal catheter system 340 into second renal vein 366. Sensing mechanisms 320, 350 can be used to detect one or more physiological parameters within the patient. For example, first sensing member 322 can be used to detect a physiological parameter of first renal artery 360, second sensing member 324 can be used to detect a physiological parameter of second renal artery 362, first sensing member 352 can be used to detect a physiological parameter of first renal vein 364, and second sensing member 354 can be used to detect a physiological parameter of second renal vein 366. It is possible to assess a physiological profile of the patient based on the physiological parameter of first renal artery 360, on the physiological parameter of second renal artery 362, on the physiological parameter of first renal vein 364, or on the physiological parameter of second renal vein 366. Similarly, it is possible to assess a physiological profile of the patient based on a combination or permutation of any of these physiological parameters.

Thus, although often catheter configurations may be primarily intended for placement within the renal arteries, there are instances where measurements or evaluations from the venous circulation may be necessary or desired to obtain, for example, differential measurements between arterial and venous circulation. Hence a second bifurcated catheter may be placed within the renal veins. A venous catheter system can determine or sense a level of excretion of a certain compound, molecule, or ion by one or both kidneys from circulation or other parameters relevant to relative renal function. In addition to obtaining differential measurements between arterial and venous locations, systems and methods embodiments of the present invention may be employed to obtain differential measurements between two or more arterial locations, such as between a first renal artery and a second renal artery, as well as to obtain differential measurements between two or more venous locations, such as between a first renal vein and a second renal vein.

An operator can use system 300 to deliver a fluid, agent, or other material to the patient, via infusion pump 336 and at least one of the first infusion port 317 and the second infusion port 319. Often, the amount or type of material administered to the patient is based on the physiological profile of the patient, as assessed or determined by module system 332. For example, use of renal catheter system 300 allows an operator to obtain a real-time assessment of renal function and other related physiological parameters relevant to the renal arteries and veins. Relatedly, an operator can use renal catheter system 300 to deliver solutions and other materials directly to the renal arteries in addition to sensing physiological parameters within or near the renal arteries and veins. Catheter system 300 can provide an operator or user with a real-time assessment or evaluation of any effects of an infusing solution, which may be given as part of a targeted renal therapy treatment. Hence, the operator can use system 300 to evaluate physiological parameters of interest, which may or may not change in response to targeted renal therapy or other surgical or medical interventions that are performed on the patient. In this way, the operator can enjoy real-time monitoring and evaluation of the efficacy and safety of targeted renal therapy, or other interventions, in light of such physiological parameters. Real-time monitoring of physiological parameters allows the user or operator to make adjustments in drugs or dosages to achieve or pursue desired pharmacological benefits. An operator can also use system 300 to obtain real-time assessment and control or modulation of renal function or related physiological parameters, during times when infusion through the catheter may or may not occur. It is appreciated that sensing members may be embedded within a catheter, and may be located on one or more branches of a catheter. In cases where simultaneous, or substantially simultaneous, and independent measurement of function of both kidneys may be desired, sensing elements may be located on both catheter branches of a bifurcated catheter. Hence, an operator can use bifurcated renal catheter system 300, which may include a monitoring or control system or module, to display and provide feedback regarding physiological parameters of interest.

Thus, in an exemplary method or procedure, an operator may assess or evaluate a physiological profile of a patient by advancing a catheter shaft of an arterial bifurcated renal catheter system into a descending aorta, such as a thoracic aorta or abdominal aorta of the patient, and advancing a catheter shaft of a venous bifurcated renal catheter system into an inferior vena cava of the patient. The operator can deploy a first catheter branch of the arterial bifurcated renal catheter system into or toward a first renal artery of the patient, and a second catheter branch of the arterial bifurcated renal catheter system into a second renal artery of the patient. The operator can also deploy a first catheter branch of the venous bifurcated renal catheter system into a first renal vein of the patient, and a second catheter branch of the venous bifurcated renal catheter system into a second renal vein of the patient. Using the catheter system, it is then possible to detect a physiological parameter of the first renal artery, and optionally a physiological parameter of the second renal artery, with a sensing mechanism of the arterial bifurcated renal catheter system, and to detect a physiological parameter of the first renal vein, and optionally a physiological parameter of the second renal vein, with a sensing mechanism of the venous bifurcated renal catheter system. The operator may, with the assistance of a module system, assessing a physiological profile of the patient based on the physiological parameter of the first renal artery, on the physiological parameter of the second renal artery, on the physiological parameter of the first renal vein, on the physiological parameter of the second renal vein, or on a combination or permutation of any of these physiological parameters.

FIG. 3B shows aspects of a renal catheter system according to embodiments of the present invention. A renal catheter system can include a bifurcated renal catheter system 370, which in turn includes a catheter 372 with a shaft 374. Catheter system 370 also includes a first catheter branch 376 and a second catheter branch 378, each coupled with catheter shaft 374. Catheter system 370 can include a delivery wire 380 coupled with one or more sensing members 382, and can also include a guide sheath 384 that is configured to receive catheter shaft 374. As shown here, first catheter branch 376 is disposed within a first renal artery 386, and second catheter branch 378 is disposed within a second renal artery 388. The renal catheter system can be used to deliver or place a sensing member in a desired location within the patient's body. For example, by retracting or advancing delivery wire 380 relative to the catheter branch, sensing member 382 can be moved in a proximal or distal direction within a renal artery.

1. Applications for Monitoring of Physiological Parameters

A. Renal Artery Vasodilation

Biological markers indicative of vasodilation, such as angiotensin II, nitric oxide (NO) or nitric oxide synthase (NOS) may be monitored via biochemical sensors specific to the detection of these markers. In some embodiments, these sensing members or sensors may be coupled with or embedded within one or more catheter branches. For example, one or more sensors may be embedded within a distal portion of a catheter branch. Detection or measurement of biomarkers specific for vasodilation that are present in the arterial circulation may be compared to detection or measurement of biomarkers that are present in the venous circulation. Such techniques can be achieved by placing a sensing bifurcated catheter platform within the renal veins. A first catheter branch of the venous catheter can be deployed at, toward, or into a first renal vein, and a second catheter branch of the venous catheter can be deployed at, toward, or into a second renal vein. In this way, an operator can assess the relative magnitude of vasodilation of the renal arteries compared to systemic circulation as a measure of GFR. It is possible to monitor or evaluate the relative effects of targeted renal therapy on vasodilation locally within the renal arteries, as opposed to the general systemic circulation, by taking comparative measurement of vasodilatory markers in renal arteries and renal veins. For example, a high differential measurement, where the concentration of a vasodilatory marker in a renal artery is much higher than the concentration of the vasodilatory marker in a renal vein, may indicate increased propensity for renal artery flow, and thus, GFR. Alternatively, a relative assessment may be made between one or more sensors disposed in the renal arteries, for example via the distal tips of a bifurcated catheter platform, and one or more sensors disposed in another artery not distally branched from the renal arteries, such as the aorta, the iliac arteries, the femoral arteries, and the like. Such a second set of measurements within arterial circulation may be achieved with the same arterial bifurcated catheter platform with sensors embedded within the catheter bifurcation base or sheath tip.

Assessment of renal artery vessel dilation may be achieved via artery luminal diameter measurements. Such measurements may provide a clinician, for example, with insight into any effects of one or more medications or administered agents on renal vasodilation. Similarly, such measurements may provide information regarding the magnitude of blood flow through the renal arteries. This information can be useful in situations where any effects of a drug, a compound, a surgical procedure, or any other intervention on renal artery vasodilation may be unknown or uncertain. In addition, in a case where an endpoint of a procedure, such as a targeted renal therapy protocol, may be increased renal artery luminal diameter, a real-time assessment of this parameter allows for instantaneous feedback regarding the effectiveness of the procedure and opportunity for adjustments to dosages or drug administration as necessary or desired to optimize or modulate any benefits or effects of the procedure. In some embodiments, measurement or detection of vessel dilation, or luminal diameter, may be achieved via an ultrasonic transducer sensor. For example, one or more ultrasonic transducer sensors may be embedded within a catheter shaft or branch of a catheter system. Optionally, a sensor may be embedded in a distal tip of a catheter branch. In some embodiments, a sensing mechanism or member may include an expandable and retractable frame.

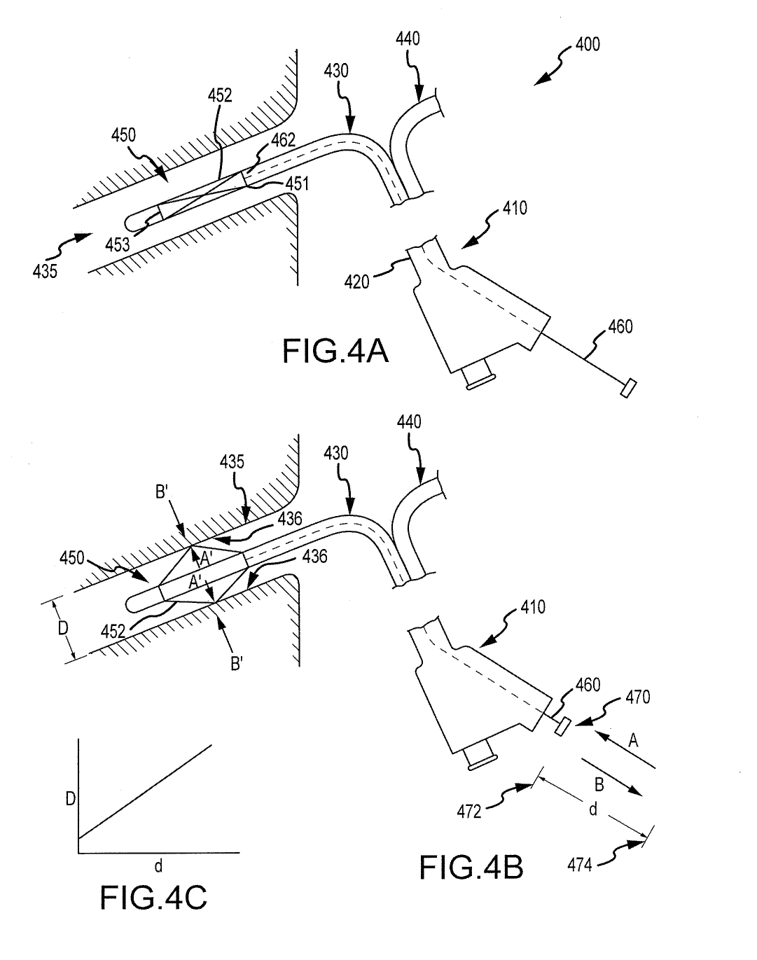

As seen in FIG. 4A, a bifurcated renal catheter system 400 can include a catheter 410 having a shaft 420 coupled with a first catheter branch 430 and a second catheter branch 440. Catheter system 400 also includes a sensing mechanism 450 for sensing or detecting physiological parameters at or near a first renal artery 435 or other vessel or location in a patient. In the embodiment shown here, sensing mechanism 450 includes a frame 452 that can be expanded and retracted. For example, the expansion and retraction of frame 452 may be facilitated by the use of a control wire 460. In some cases, frame 452 comprises a metal material. FIG. 4A shows frame 452 in a retracted configuration, and FIG. 4B shows frame 452 in an expanded configuration. As depicted in FIG. 4B, control wire 460 can be coupled with frame 452, such that when control wire 460 is advanced in a distal direction relative to first catheter branch 430, as indicated by arrow A, frame 452 adopts a first configuration and is expanded radially from first catheter branch 430, as indicated by arrow A′. Relatedly, when control wire 460 is withdrawn in a proximal direction relative to first catheter branch 430, as indicated by arrow B, frame 452 adopts a second configuration and is retracted radially toward first catheter branch 430, as indicated by arrow B′. A proximal end 462 of control wire 460 can be coupled with a proximal ring or sliding mechanism 451 of frame 452. Proximal sliding mechanism 451 can be configured to slide or translate along a length of catheter branch 430. Frame 452 may also include a distal ring or fixed mechanism 453 that is affixed to or otherwise remains stationary relative to catheter branch 430.

It is possible to determine or evaluate a diameter or other dimension D of first renal artery 435 using frame 452 and control wire 460. For example, as control wire 460 is pushed or advanced proximally, it induces the expansion or opening of frame 452. After control wire 460 is advanced a certain distance d, frame 452 is expanded sufficiently such that the frame contacts the inner wall of the artery. Hence, frame 452 becomes expanded to match or approximate diameter or dimension D of the renal artery. In this way, by determining a distance d that control wire is moved, it is possible to calculate the diameter or other dimension D of the renal artery. If control wire 460 moves only a slight distance d in the proximal direction until frame 452 contacts the artery wall, it can be determined that the diameter D of the artery wall is relatively small. Conversely, if control wire 460 moves a longer distance d in the proximal direction until frame 452 contacts the artery wall, it can be determined that the diameter D of the artery wall is relatively large. Movement of the control wire can be actuated either manually or automatically. In either instance, the distance the control wire has been advanced may be used to determine the diameter of the renal artery.

FIG. 4C provides a graphic representation of a relationship between control wire movement distance d and arterial or frame diameter D. As shown here, the distance d is proportional to the amount of expansion of the frame. With reference to FIG. 4B, it is possible to measure distance d by measuring the distance a control wire hub 470 moves during operation of the catheter. For example, distance d may represent a distance between a proximal location 472 of control wire hub 470, where frame 452 is in an expanded configuration and in contact with the artery wall 436, and a distal location 474 of control wire hum 470, where frame 452 is in a contracted or collapsed configuration. In the manner described above, system 400 can be used to evaluate the degree to which a renal artery is vasodilated, and hence can be used to assess a physiological profile of a patient. It is understood that although FIGS. 4A and 4B discuss a vasodilation measurement mechanism for first renal artery 430, system 400 may also include complementary elements for a vasodilation measurement mechanism associated with second catheter branch 440, for evaluating a physiological parameter of a second renal artery. Moreover, system 400 may include aspects of catheter system embodiments disclosed elsewhere herein. For example, system 400 may include a module system or a second venous bifurcated catheter system.

B. Renal Blood Flow

Measurement of the magnitude of blood flow through the renal arteries may be achieved via monitoring of physical parameters such as volumetric flow rate and inner arterial wall shear stress. Magnitude of blood flow may provide the clinician with information towards the degree of oxygenation and nutrition of the kidneys, in addition to serving as an indicator for GFR. This information can be particularly useful in situations where any effect of a drug, a compound, a surgical procedure, or other intervention on renal blood flow may be unknown or uncertain. In addition, in a case where an endpoint of a procedure, such as a targeted renal therapy protocol, may involve renal blood flow, a real-time assessment of this parameter allows for instantaneous feedback regarding the effectiveness of the procedure and opportunity for adjustments to dosages or drug administration as necessary or desired to optimize or modulate any benefits or effects of the procedure.

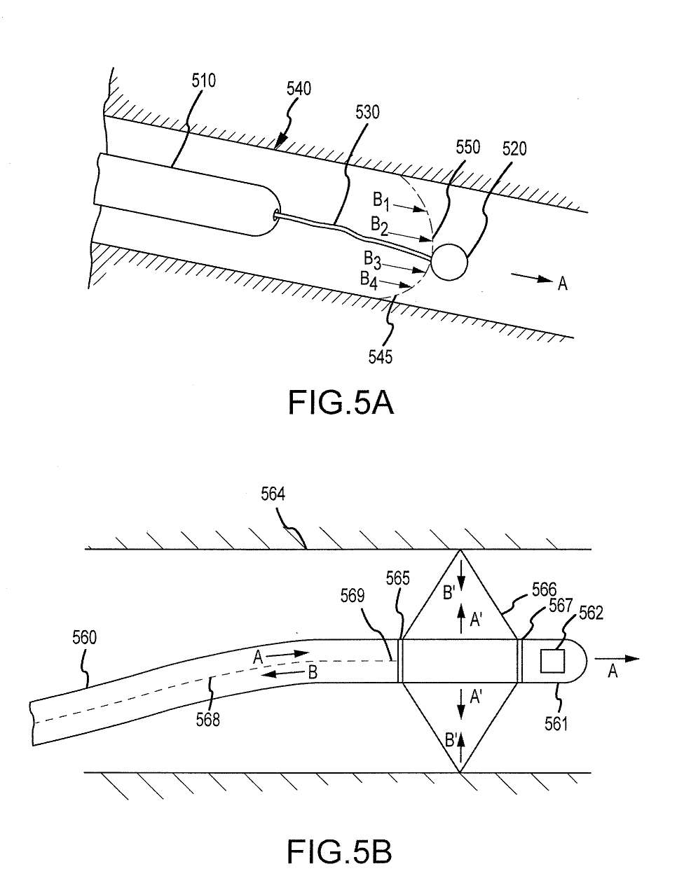

Peak flow velocity within a luminal cross section where monitored can be used to derive total volumetric flow. To measure peak flow velocity, a sensor can be positioned accurately at such location. In one technique according to embodiments of the present invention, a flow-guided sensor can be used to find a position within a renal artery or other lumen corresponding to peak flow velocity. As shown in FIG. 5A, a catheter branch 510 of a catheter system can be coupled with a sensor 520 via a tether 530. Blood can flow through renal artery 540 in the direction indicated by arrow A. An intra-renal flow profile 545 is indicated by arrows B₁₋₄, where higher flow velocities are represented by longer arrows and lower flow velocities are represented by shorter arrows. As shown here, the peak flow velocity 550 of flow profile 545 occurs toward the center of renal artery 540. As blood flows past sensor 520, the flow rate sensor adjusts itself by way of imposed flow shear stresses towards the position of peak flow velocity. Flow is often measured via shear stress sensors that measure the drag imposed on a plate placed parallel to a flow's streamlines. In some cases, a sensor may include a MEMS shear stress sensors or the like. Such sensors are discussed in Soundararajana et al., “MEMS Shear Stress Sensors for Microcirculation,” Sensors and Actuators A: Physical, Volume 118, Issue 1, 31 Jan., Pages 25-32 (2005), the contents of which are incorporated herein by reference. In some cases, a sensor may incorporate Doppler technology, such as those marketed by Volcano Corporation (San Diego, Calif.).

In another technique according to embodiments of the present invention, a sensing mechanism may include a positioning mechanism that can be used to position a sensing member in a desired location within the patient's body. For example, a sensing mechanism may include an expandable and retractable frame or a balloon incorporated on or coupled with a catheter branch. The frame or balloon can be used to center or otherwise position a sensor at a location within the renal artery lumen. As depicted in FIG. 5B, a catheter branch 560 of a catheter system can be coupled with a flow rate sensor 562. Similar to the configurations described in FIGS. 4A and 4B, catheter branch 560 of FIG. 5B may be coupled with an expandable and retractable frame 566 which can operate here as a positioning mechanism for the flow rate sensor. The expansion and retraction of frame 566 may be facilitated by the use of a control wire 568. In some cases, frame 566 comprises a metal material. FIG. 5B shows frame 566 in an expanded configuration. Control wire 568 can be coupled with frame 566, such that when control wire 568 is advanced in a distal direction relative to first catheter branch 560, as indicated by arrow A, frame 566 adopts a first configuration and is expanded radially from first catheter branch 560, as indicated by arrow A′. Relatedly, when control wire 568 is withdrawn in a proximal direction relative to first catheter branch 560, as indicated by arrow B, frame 566 adopts a second configuration and is retracted radially toward first catheter branch 560, as indicated by arrow B′. A proximal end 569 of control wire 568 can be coupled with a proximal ring or sliding mechanism 565 of frame 566. Proximal sliding mechanism 565 can be configured to slide or translate along a length of catheter branch 560. Frame 566 may also include a distal ring or fixed mechanism 567 that is affixed to or otherwise remains stationary relative to catheter branch 560. Flow rate sensor 562 may be embedded in a distal tip 561 of catheter branch 560. In some embodiments, flow mechanics principles may consider the center of a flow profile to coincide with a point of peak flow. Frame 566 can be configured so as to position sensor 562 at any desired location within the renal artery, which in some cases may be at a point of peak flow. Accordingly, frame 566 can be configured to position sensor 562 toward the center of the renal artery when the frame is deployed or expanded. In some embodiments of the present invention, total or volumetric flow within a renal artery may be derived using a combined set of measurements for peak flow velocity and luminal diameter via techniques described herein.

In some embodiments, a sensing mechanism may include a balloon assembly for positioning a sensor or sensing member at a location within the patient. A balloon assembly may have a multi-prong configuration that allows for the passage of blood past a branch-integrated deployed balloon. Relatedly, in some cases a balloon assembly may be disposed or integrated with a catheter shaft. In some cases, a positioning assembly can be used to position an infusion port. FIG. 5C shows an axial cross-sectional view of a balloon assembly according to embodiments of the present invention. In this multi-prong configuration, balloon assembly 570 is expanded so that the outer portions 572 of first balloon 574 and second balloon 576 contact the interior wall of a lumen or vessel 578, such as a renal artery. First balloon 574 is in fluid communication with an inflation lumen 577 of catheter branch 571 via a first inflation port 573, and second balloon 574 is in fluid communication with inflation lumen 577 of catheter branch 571 via a second inflation port 575. As shown here, there can be a 180° offset between the expanded lobe of first balloon 574 and the expanded lobe of second balloon 576. Other offset configurations may be employed. One or more sensors 579 may be coupled with or embedded within branch 571 or balloons 574, 576 at desired locations, such that when the balloon assembly is advanced into a vessel or lumen and expanded, the sensor 579 can be positioned at or near a specific target area within the cross-section of the vessel or lumen.

FIG. 5D shows an axial cross-sectional view of a balloon assembly according to embodiments of the present invention. In this multi-prong configuration, balloon assembly 580 is expanded so that the outer portions 582 of first balloon 584a, second balloon 584b, and third balloon 584c contact the interior wall of a lumen or vessel 588, such as a renal artery. First balloon 584a is in fluid communication with an inflation lumen 587 of catheter branch 581 via a first inflation port 583a, second balloon 584b is in fluid communication with inflation lumen 587 of catheter branch 581 via a second inflation port 583b, and third balloon 584c is in fluid communication with inflation lumen 587 of catheter branch 581 via a third inflation port 583c. As shown here, there can be a 120° offset between the expanded lobe of first balloon 584a and the expanded lobe of second balloon 584b, a 120° offset between the expanded lobe of second balloon 584b and the expanded lobe of third balloon 584c, and a 120° offset between the expanded lobe of third balloon 584c and the expanded lobe of first balloon 584a. Other offset configurations may be employed. One or more sensors 589 may be coupled with or embedded within branch 581 or balloons 584a, 584b, 584c at desired locations, such that when the balloon assembly is advanced into a vessel or lumen and expanded, the sensor 589 can be positioned at or near a specific target area within the cross-section of the vessel or lumen.

FIG. 5E shows an axial cross-sectional view of a balloon assembly according to embodiments of the present invention. In this multi-prong configuration, balloon assembly 590 is expanded so that the outer portions 592 of first balloon 594a, second balloon 594b, third balloon 594c, and fourth balloon 594d contact the interior wall of a lumen or vessel 598, such as a renal artery. First balloon 594a is in fluid communication with an inflation lumen 597 of catheter branch 591 via a first inflation port 593a, second balloon 594b is in fluid communication with inflation lumen 597 of catheter branch 591 via a second inflation port 593b, third balloon 594c is in fluid communication with inflation lumen 597 of catheter branch 591 via a third inflation port 593c, and fourth balloon 594d is in fluid communication with inflation lumen 597 of catheter branch 591 via a third inflation port 593d. As shown here, there can be a 90° offset between the expanded lobe of first balloon 594a and the expanded lobe of second balloon 594b, a 90° offset between the expanded lobe of second balloon 594b and the expanded lobe of third balloon 594c, a 90° offset between the expanded lobe of third balloon 594c and the expanded lobe of fourth balloon 594d, and a 90° offset between the expanded lobe of fourth balloon 594d and the expanded lobe of first balloon 594a. Other offset configurations may be employed. One or more sensors 599 may be coupled with or embedded within branch 591 or balloons 594a, 594b, 594c, 594d at desired locations, such that when the balloon assembly is advanced into a vessel or lumen and expanded, the sensor 599 can be positioned at or near a specific target area within the cross-section of the vessel or lumen.

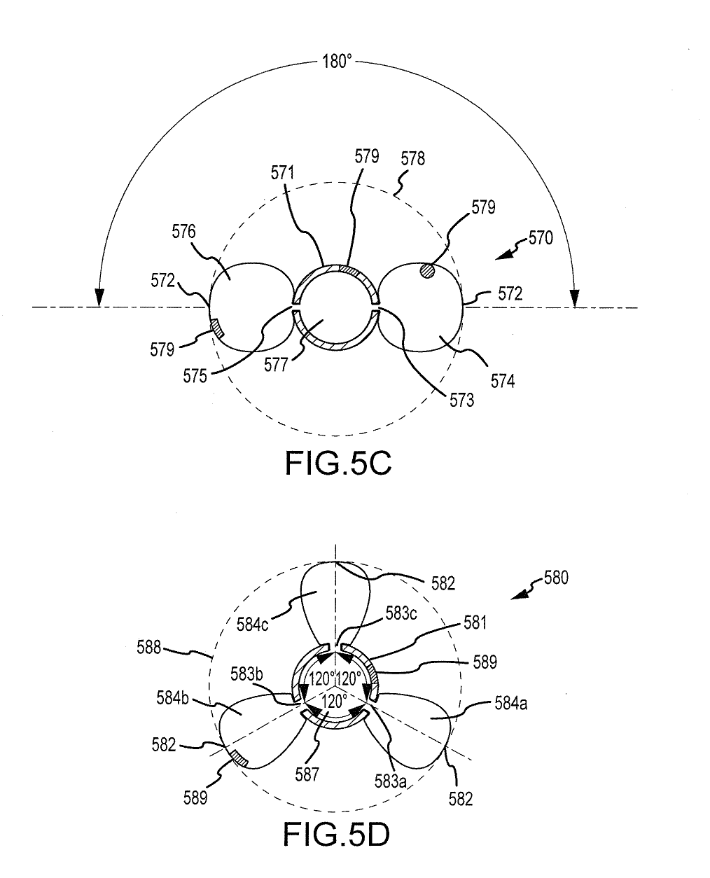

In another embodiment, volumetric renal artery blood flow may be measured via drag force measurement of a deployed mesh umbrella or parachute that is coupled with a catheter branch. FIG. 6 illustrates a drag force measurement assembly 610 according to embodiments of the present invention. Assembly 610 is coupled with a catheter branch 605 of a catheter system. As shown here, catheter branch 605 can be placed within a vessel or lumen 607, such as a renal artery. Assembly 610 can include a drag mechanism 612, which may include a mesh umbrella or parachute or any other type of net, sieve, or screen that allows fluid to pass therethrough. Drag mechanism 612 may in some cases include a solid object that generates drag in the fluid. Friction generated between the flowing fluid and the drag mechanism 612 can result in a drag force. A mesh umbrella or sieve can be constructed of or include a flexible material, such as nylon, PET or PTFE, that facilitates reliable deployment and retraction of the drag mechanism, while allowing for the drag mechanism to reasonably conform to the varying shapes and contours of the renal artery lumen. Once deployed, a branch tip-suspended mesh structure can be pulled by blood flow away from the catheter branch. The resulting drag force imposed on the catheter branch via the drag mechanism, which can be correlated to the volumetric renal artery blood flow rate or total flow momentum, can be measured via a force transducer 614 coupled with drag mechanism 612. In use, drag mechanism 612 may be deployed and undeployed via activation of a control wire 616. For example, control wire 616 may be advanced in a distal direction as indicated by arrow A, so as to deploy drag mechanism 612 away from catheter branch 605 and into the renal blood flow. Conversely, control wire 616 may be retracted in a proximal direction as indicated by arrow B, so as to undeploy drag mechanism 612 by moving the mechanism toward, and optionally into, catheter branch 605. For example, drag mechanism may be retracted into an aperture 618 disposed on catheter branch 605. In this way, drag mechanism 612 can be removed from the renal blood flow. Drag force measurement assembly 610 can be used to measure a volumetric renal artery blood flow that is flowing in the direction indicated by arrow C.

Any of a variety of flow measurement mechanisms can be used to evaluate volumetric blood flow in a patient vessel such as the renal artery. For example, magnetic flow meters, Coriolis flow meters, paddle wheel flow meters, vortex flow meters, and the like can be incorporated in a renal catheter system. Often, such flow measurement mechanisms are coupled with or embedded in one or more branch catheters of the system.

Techniques for evaluating total volumetric blood flow within the renal arteries may also be based on the measurement of luminal wall shear stress. For example, a catheter system can include a shear stress sensor that is positioned near or adjacent to an inner wall of an artery, and volumetric blood flow rate can be derived using measurements of luminal diameter, as described elsewhere herein. Total volumetric flow can be calculated for laminar flows and Newtonian fluids based on the Hagan-Poiseuille equation: Q=τ*(πR̂3/4) where τ is wall shear stress, Q is total/volumetric flow rate and R is the radius of the vessel. Hence, the combination of the vessel diameter and wall shear stress measurements can allow for computation of total volumetric flow. Embodiments of the present invention encompass a variety of approaches for delivering a shear stress sensor to the wall of a renal artery.

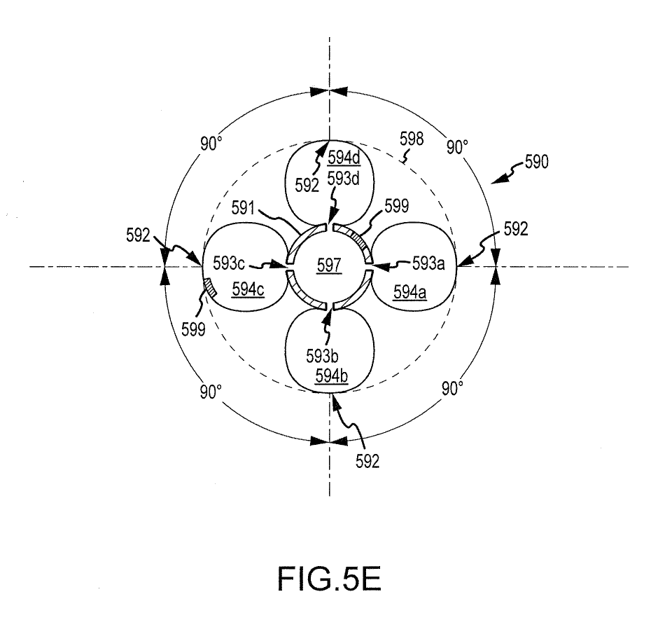

As shown in FIG. 7, an expandable and retractable frame having a sensor can be coupled with a catheter branch. Hence, this sensing mechanism includes a positioning mechanism that can be used to position the sensing member in a desired location within the patient's body. The sensing mechanism may include an expandable and retractable frame incorporated on or coupled with a catheter branch. The frame can be used to position a sensor at a location within the renal artery lumen. A catheter branch 710 of a catheter system can be coupled with a shear stress sensor 720, for example via an expandable and retractable frame 730. Similar to the configurations described in FIGS. 4A and 4B, and FIG. 5B, catheter branch 710 may be coupled with an expandable and retractable frame 730 which can operate here as a positioning mechanism for the shear stress sensor. The expansion and retraction of frame 730 may be facilitated by the use of a control wire 740. In some cases, frame 730 comprises a metal material. FIG. 7 shows frame 730 in an expanded configuration. Control wire 740 can be coupled with frame 730, such that when control wire 740 is advanced in a distal direction relative to catheter branch 710, as indicated by arrow A, frame 730 adopts a first configuration and is expanded radially from catheter branch 710, as indicated by arrow A′. Relatedly, when control wire 740 is withdrawn in a proximal direction relative to catheter branch 710, as indicated by arrow B, frame 730 adopts a second configuration and is retracted radially toward catheter branch 710, as indicated by arrow B′. A proximal end 749 of control wire 740 can be coupled with a proximal ring or sliding mechanism 732 of frame 730. Proximal sliding mechanism 732 can be configured to slide or translate along a length of catheter branch 710. Frame 730 may also include a distal ring or fixed mechanism 734 that is affixed to or otherwise remains stationary relative to catheter branch 710. Shear stress sensor 720 may be embedded in or coupled with an arm 736 of frame 730. Frame 730 can be configured so as to position sensor 720 at any desired location within the renal artery, which in some cases may be at or near an interior wall 752 of a vessel or lumen 750, such as a renal artery. Accordingly, frame 730 can be configured to position sensor 720 toward the renal artery wall when the frame is deployed or expanded. In some embodiments of the present invention, total or volumetric flow within a renal artery may be derived using a combined set of measurements for shear stress and luminal diameter via techniques described herein. As shown here, the catheter system can include a sensor wire or cable 722 that transmits sensor data or signals to and from sensor 720. Hence, a frame on a catheter branch can carry a mounted sensor on the periphery of the frame. Upon deployment of the frame structure, the sensor can be brought in contact with the inner wall of the artery.

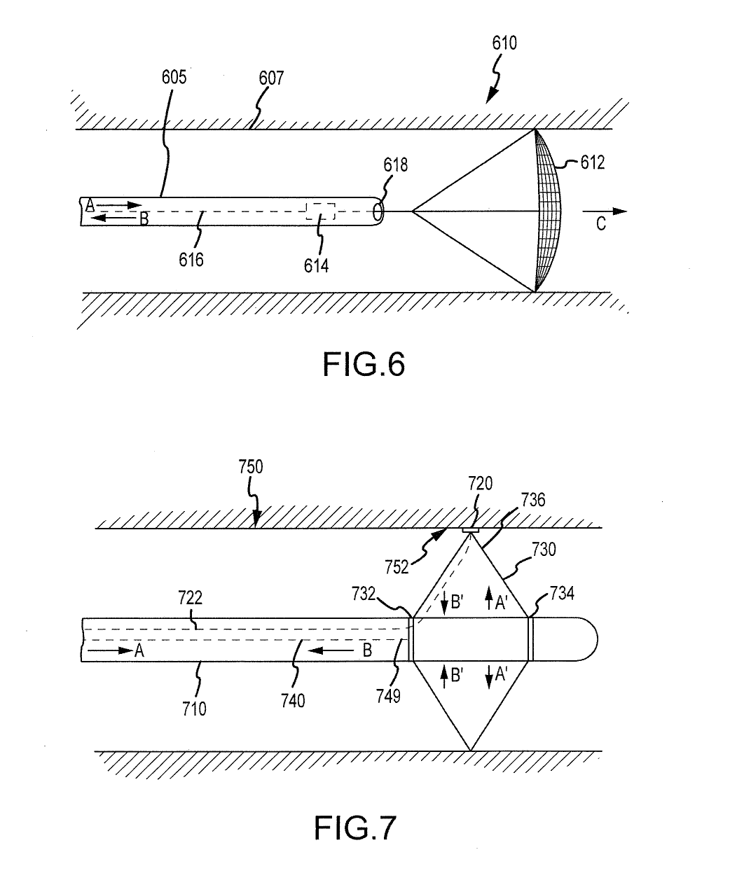

As shown in FIG. 8, a balloon assembly having a releasable sensor can be coupled with a catheter branch. Hence, this sensing mechanism includes a positioning mechanism that can be used to position and implant the sensing member in a desired location within the patient's body. The sensing mechanism may include an expandable and retractable balloon assembly incorporated on or coupled with a catheter branch. The balloon assembly can be used to position a sensor at a location within the renal artery lumen. A catheter branch 810 of a catheter system can be releasably coupled with a telemetric shear stress sensor 820, for example via an expandable and retractable balloon assembly 830. Upon deployment of the balloon assembly, the sensor can be brought in contact with an inner wall of an artery. The catheter system can be configured to allow for the passage of blood flow during deployment of a balloon assembly. Telemetry-configured shear stress sensor 820 may be placed as an implant on the inner wall 852 of a vessel or lumen 850, such as a renal artery. The telemetry configuration can provide the ability for wireless monitoring of sensor measurements. Any of the expandable frame or balloon assemblies described herein can be used to deliver the sensor to the artery wall. As shown in FIG. 8, sensor 820 may be affixed to artery wall 852 via anchors 822, an adhesive such as fibrin glue or cyanoacrylate, or any combination thereof. In some embodiments, a balloon 832 of balloon assembly 830 can include pores 834 that deliver an adhesive material. Hence, using the balloon assembly, an adhesive may be placed on the sensor by inflating the balloon with the desired adhesive. Pores on the balloon concentrated or located around the sensor can release the adhesive material on the sensor surface. In some embodiments, an adhesive glue can be used to maintain the implanted sensor. The glue may be blood pH-activated, and the sensor can be held at the wall of the artery for a certain period of time to allow for glue polymerization. One or more anchors 822 can promote patency on the vessel wall.

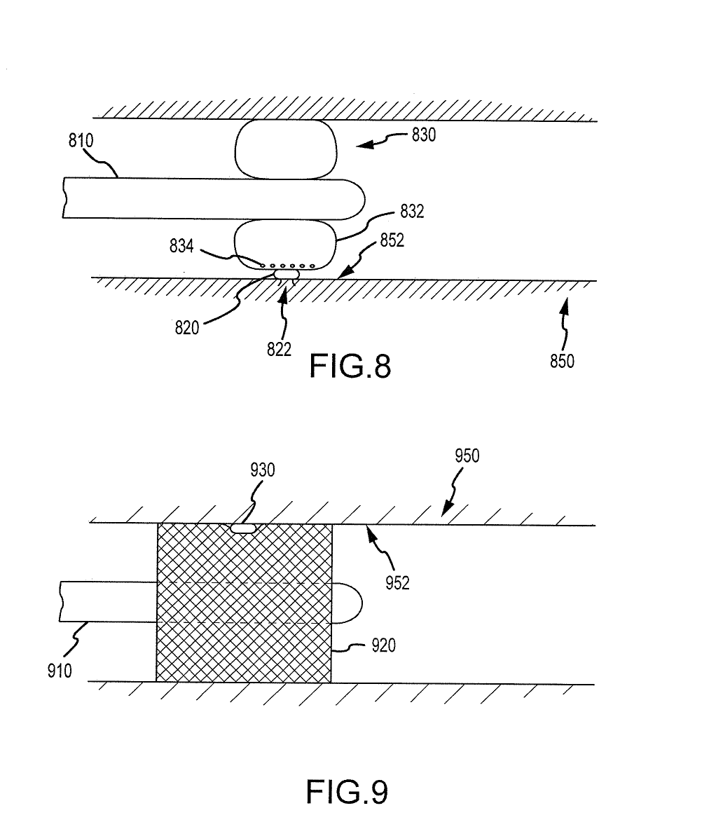

FIG. 9 shows aspects of a catheter system for delivering and implanting a sensor, according to embodiments of the present invention. An expandable stent having sensor can be coupled with a catheter branch. Hence, this sensing mechanism includes a positioning mechanism that can be used to position and implant the sensing member in a desired location within the patient's body. The sensing mechanism may include an expandable stent incorporated on or releasably coupled with a catheter branch. The stent can be used to position a sensor at a location within the renal artery lumen. A catheter branch 910 of a catheter system can be releasably coupled with stent 920, and stent 920 may be coupled with a telemetric shear stress sensor 930. Upon deployment of the stent, the sensor can be brought in contact with an inner wall of an artery. The catheter system can be configured to allow for the passage of blood flow during deployment the stent. Telemetry-configured shear stress sensor 930 may be placed as an implant on the inner wall 952 of a vessel or lumen 950, such as a renal artery. The telemetry configuration can provide the ability for wireless monitoring of sensor measurements. The stent can be deployed and implanted into the vessel or lumen, and upon deployment of the stent the shear stress sensor can be brought into contact with and implanted against the surface of the vessel or lumen wall.

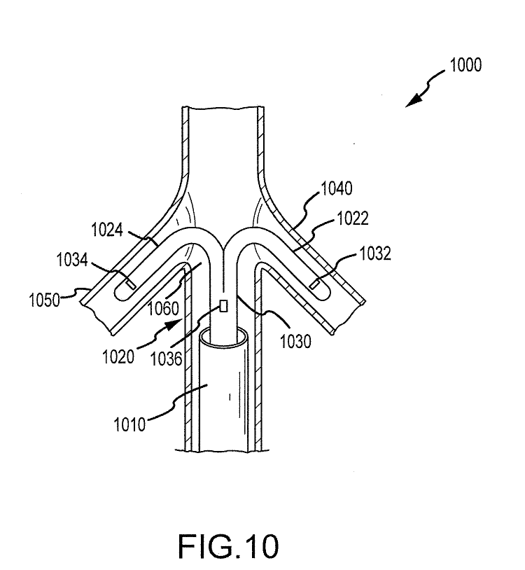

According to some embodiments of the present invention, evaluation of renal blood flow can be performed based on blood pressure measurements. FIG. 10 shows a catheter system 1000 that can be used to derive total renal blood flow. Catheter system 1000 includes a guide sheath 1010, and a catheter 1020 having a shaft 1030. Catheter 1020 also includes a first catheter branch 1022 and a second catheter branch 1024 coupled with catheter shaft 1030. Catheter system 1000 can also include pressure sensors located at a variety of positions on the system. For example, pressure sensor 1032 can be located on first catheter branch, pressure sensor 1034 can be located on second catheter branch, and pressure sensor 1036 can be located on catheter shaft 1030. In some embodiments, a pressure sensor can be located on a guide sheath. Pressure sensor 1032 can measure a pressure within a first renal artery 1040 of the patient, pressure sensor 1034 can measure a pressure within a second renal artery 1050 of the patient, and pressure sensor 1036 can measure a pressure within an aorta 1060 of the patient, for example in a descending aorta at or near the level of the renal arteries. In conjunction with renal artery diameter measurements, which may be obtained pursuant to diameter evaluation techniques described herein, pressure measurements can be used to provide an assessment of mechanical flow resistance within a renal artery.

Since the sensor locations are fixed length is constant and resistance only is a function of luminal diameter, the difference in pressure between these two locations may be used to derive the volumetric flow within the renal arteries. Total volumetric flow can be calculated for laminar flows and Newtonian fluids based on the Hagan-Poiseuille equation: Q=π*R̂4*(P₁−P₂)/(2*L) where P₁ and P₂ are pressures at two arbitrary points, Q is total/volumetric flow rate, R is the radius of the vessel and L is the axial distance between these 2 arbitrary points. As shown in FIG. 10, multiple sensors may be placed on renal catheter system 1000 to yield such pressure measurements. A sensor associated with the catheter shaft, for example sensor 1036, can measure aortic blood pressure. Sensors associated with the catheter branches, for example sensors 1032, 1034, can measure renal artery pressure.

According to some embodiments of the present invention, renal artery blood flow can be determined via differential pressure measurements between the renal artery and that of venous circulation. This approach involves using a measured diameter of the renal artery, for example by a diameter measurement technique described herein, as an estimation of relative changes in the resistance of the renal vascular bed. Relative changes to renal artery volumetric blood flow rate may be assessed using combinations of pressure measurements within the renal arteries, for example as detected by sensors on catheter branches of an arterial bifurcated renal catheter system, pressure measurements within the venous circulation, for example at or near the renal veins as detected by sensors on catheter branches of a venous bifurcated renal catheter system, and the renal artery luminal diameter measurements. This can be calculated based on Poiseuille's equation for laminar Newtonian flow: Q=ΔP*(πr⁴)/(81μ) where ΔP is the pressure difference between two arbitrary points in a vessel, r is vessel radius, 1 is the length or axial distance between those two arbitrary points, and μ is blood viscosity. In this way, blood pressure measurements can be used to derive total renal blood flow. Catheter systems such as those disclosed with reference to FIG. 3A, for example, are well suited for use in such techniques.

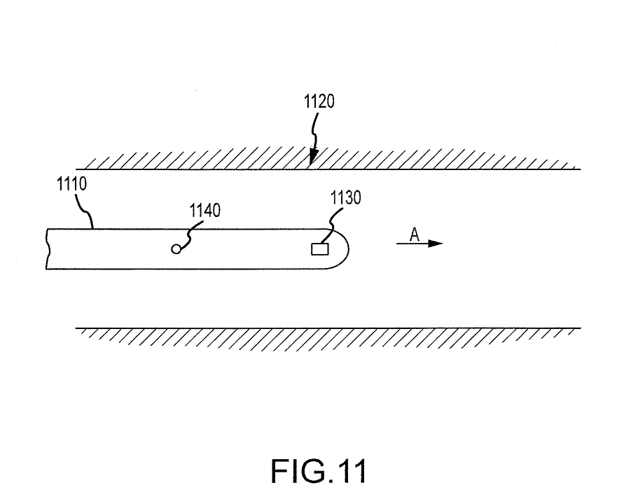

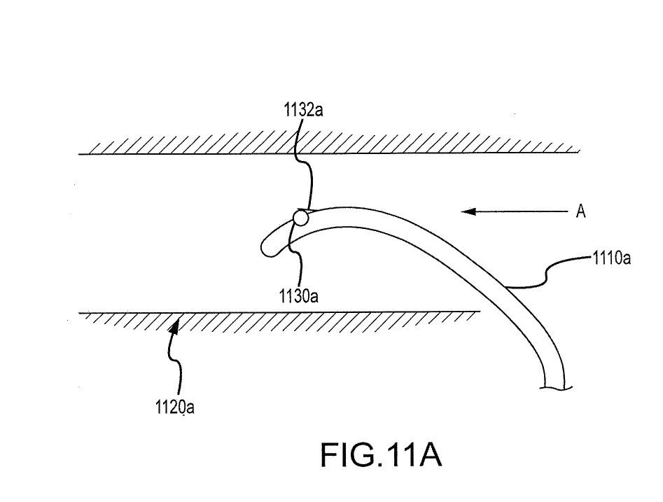

FIG. 11 depicts features of a renal catheter system that can utilize the principle of thermal dilution to derive renal artery blood flow, according to embodiments of the present invention. The renal catheter system includes a renal catheter branch 1110 that can be deployed into a renal artery 1120. The renal catheter system also includes a temperature sensor 1130, such as a thermocouple, a thermistor, a resistance temperature detector, or the like, embedded within or coupled to catheter branch 1110. Further, the renal catheter system includes an infusion or injection port 1140, for example disposed on or coupled with catheter branch 1110. As shown here, temperature sensor 1130 is positioned at a location distal to infusion or injection port 1140. In use, a fluid or solution have a temperature that is different from the patient's renal arterial blood temperature, for example a fluid at room temperature, can be introduced to a patient's renal artery 1120 via port 1140. In some cases, this may involve administering the fluid through a bifurcated renal catheter branch via a renal catheter shaft. As the fluid exits port 1140, and flows in a downstream direction as indicated by arrow A, a temperature resulting from the degree of thermal dilution fluid can be measured. For example, if the fluid is originally at room temperature (e.g. 25° C.) as it exits port 1140, and the patient's renal arterial blood is at body temperature (e.g. 37° C.), then temperature sensor 1130 may detect a cooling trend in the surrounding blood as the colder fluid mixes with the blood, and the temperature of the surrounding blood near the sensor becomes lower than body temperature. In this way, it is possible to assess volumetric flow in the patient. Aspects of such volumetric flow techniques are discussed in Pávek et al. in “Measurement of Cardiac Output by Thermodilution with Constant Rate Injection of Indicator,” Circulation Research, Vol. XV, October, pp. 311-319 (1964), the entire contents of which are incorporated herein by reference. The governing equation for deriving volumetric flow based on temperature measurement at a point distal to the point of injection is: Q=fᵢ(Tᵥ−Tᵢ)/(T_{b}−Tᵥ)*k, where Q is blood volumetric flow rate, fᵢ is the volumetric infusion rate through the catheter branch, Tᵥ is temperature measured by sensor at distal branch tip, Tᵢ is the temperature of infusing solution, T_{b} is blood temperature and k is a constant related to the specific weight and specific heat of the blood and infusing solution. This may account for algorithmic offsets due to the physical separation between the point of measurement and initial blood-infusant mixing. In some embodiments, the renal catheter system can be used to evaluate a time lag associated with the temperature sensor's detection of a temperature change, where corresponding to the advection or volumetric flow rate within the renal artery.

Volumetric blood flow can also be evaluated by using a catheter system that incorporates the principles of a Pitot tube. A Pitot device can determine a fluid flow velocity based on pressure measurements. For example, by measuring the difference between stagnation pressure and static pressure, dynamic pressure can be determined, and used to calculate volumetric blood flow. As discussed here, stagnation pressure can be the sum of static pressure and dynamic pressure. In one exemplary technique, according to embodiments of the present invention, multiple pressure sensors may be used to obtain differential pressure measurements. For example, a first pressure sensor can be disposed on a catheter branch. This sensing member can act as a sensor for stagnation pressure, and can be oriented on the catheter branch toward/against the renal blood flow. A second pressure sensor can also be disposed on the catheter branch. This sensing member can act as a sensor for static pressure, and can be oriented away from or perpendicular to renal blood flow. Such a dual pressure sensor configuration can provide a user or operator with a dynamic pressure measurement of renal blood flow. FIG. 11A depicts features of a renal catheter system that can utilize the principles of pressure sensing, according to embodiments of the present invention. The renal catheter system includes a renal catheter branch 1110a that can be deployed or placed into a renal artery 1120a. The renal catheter system also includes a static pressure sensor 1130a embedded within or coupled to catheter branch 1110a. The system may also include a shield 1132a that can be placed over a proximal face or surface of the pressure sensor. The shield can prevent or inhibit direct contact between a flow streamline and therefore provide a stagnation pressure reading. The static pressure sensor can be housed at least partially within the shield, so that pressure measurements can be made while the pressure sensor is protected from direct contact with flow streamlines. Arrow A represents the direction of renal blood flow within renal artery 1120a. As shown here, the static pressure sensor can be disposed at a downstream location relative to the shield.

In some embodiments, renal blood flow measurements can be determined by evaluating physiological biological markers such as acetylcholine and bradykinin. Such biological markers may be monitored via biochemical sensors, for example by sensors embedded within the catheter branch tips of the a bifurcated renal catheter system. In addition, measurement of aldosterone, a marker indicating changes to blood pressure, via marker-specific biochemical sensors may provide another basis for assessing renal blood flow. These markers may be measured at two or more different points within the patient's circulation to allow for derivation of renal blood flow. For example, a primary measurement may be made within the renal arteries. Such measurements can be determined via sensors embedded within or coupled with catheter branches of a bifurcated arterial renal catheter system. A second set of readings may originate from the aorta. Such measurements can be determined via sensors embedded within or coupled with a catheter shaft of the catheter system, or via sensors embedded within or coupled with a guide sheath of the catheter system. In some cases, a second set of readings may originate from the venous circulation. Such measurements can be determined via sensors embedded within or coupled with catheter branches of a bifurcated venous renal catheter system.

Since mechanical resistance between the pressure points can be derived for calculation of renal blood flow, renal artery luminal diameter measurements (via one or more methods previously described) may be achieved with the bifurcated catheter platform. Based on Poiseuille's equation for laminar Newtonian flow: Q=ΔP*(πr⁴)/(81μ) where ΔP is the pressure difference between two arbitrary points in a vessel, r is vessel radius, 1 is the length or axial distance between those two arbitrary points and μ is blood viscosity.

C. Renal Function: Glomerular Filtration Rate (GFR)

Measurement of physiological markers such as renin, angiotensin II, SrCr, cystanin C, urea, BUN, electrolytes (e.g. sodium, potassium, chloride, or bicarbonate), and pH via marker-specific biochemical sensors embedded within or coupled with catheter branches of a bifurcated renal catheter may provide an assessment of renal function. Such measurements within the renal arteries may be compared to additional measurements of the same markers from the venous circulation. It is possible to use such comparisons to evaluate the concentration of these markers cleared from blood by the kidneys. Hence, the differential measurements can be used to obtain a measurement of GFR, and other indicators of renal function. Likewise, catheter branch biochemical sensors specific to oxygen, reactive oxygen species (ROS), or neutrophil gelatinase-associated lipocalin (NGAL) can be used to indicate or monitor any reduced renal function or renal damage. A clinician can determine or evaluate any effects of a procedure or treatment on renal function, by monitoring physiological parameters or markers.

D. Measurement of Blood Contrast Solution Concentration

Certain diagnostic procedures and other interventions involve the administration of contrast solutions or agents to a patient. Measurements of renal artery blood sodium/calcium ion balances or pK/pH levels via biochemical sensors embedded within or coupled with catheter branches of a bifurcated renal catheter system can be used to assess the amount or concentration of a contrast solution that exists within circulating blood. A contrast solution can induce a nephrotoxic effect on the kidneys. Hence, bifurcated renal catheter systems according to embodiments of the present invention can be used to assess a clinical risk for renal damage as caused by contrast solution exposure. Such assessments of contrast solution concentration in blood may increase the efficacy of blood. The determination of targeted renal therapy management, dosage, and infusion time, for example, to deliver optimal or desired therapy or care for the kidneys via infusible bifurcated renal catheter systems, can be based on an assessment of contrast solution in blood. For example, a clinician may determine, optionally based at least in part upon output from a module system, to administer a targeted renal therapy or other intervention until such time that an injected contrast solution has been sufficiently excreted from blood circulation. Thus, a measurement of blood contrast solution concentration can provide the clinician with an indication of an appropriate time to discontinue administration of the targeted renal therapy or other intervention.

E. Lesion Analysis and Therapeutic Management of Renal Artery Stenosis

Bifurcated renal catheter systems and methods according to embodiments of the present invention can be used to measure physiological parameters relevant to the degree of effects of a renal artery stenosis on local hemodynamics. For example, a pressure sensor can act as a navigation aid for a catheter branch, in guiding the branch across a stenosis and measuring a change in pressure associated with the stenosis. In addition, a flow sensor in conjunction with a pressure sensor can provide information regarding the efficacy of a procedure intended to open the stenosis, for example by taking flow measurements before and after the procedure. In some embodiments, a renal catheter system may include a flow sensor within or coupled with a catheter branch to help assess a relative increase in renal artery blood flow after a procedure intended to open the lesion has been performed, for example by making measurements before and after the procedure.

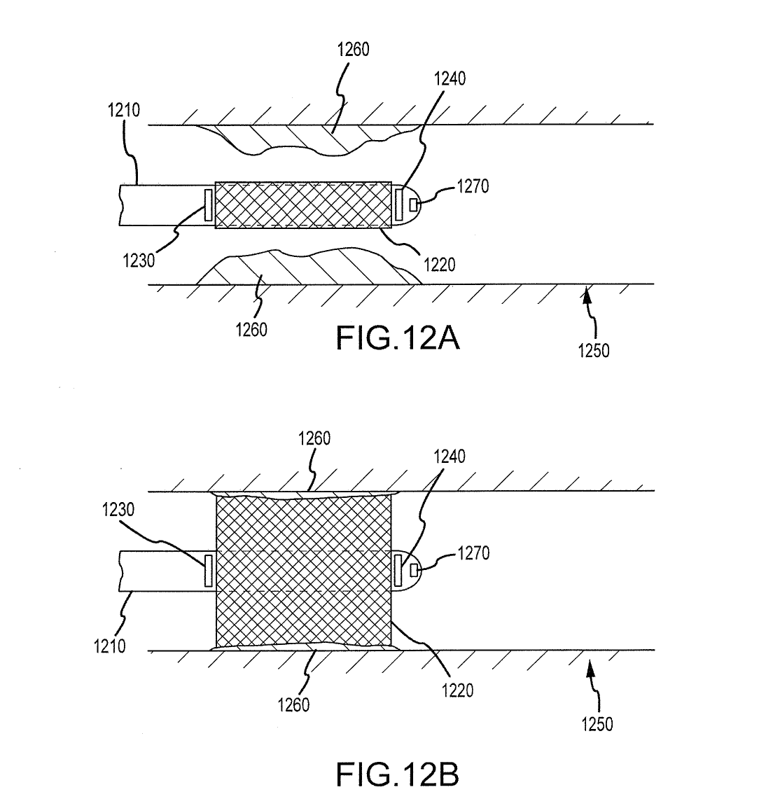

FIG. 12A shows aspects of a renal catheter system according to embodiments of the present invention. The catheter system includes a catheter branch 1210 loaded with or coupled with an expandable stent 1220 that can be released from the catheter branch. As shown here, stent 1220 is undeployed, in a collapsed or retracted configuration. Catheter branch 1210 also includes or is coupled with a proximal pressure sensor 1230 and a distal pressure sensor 1240. As shown here, proximal pressure sensor 1230 is disposed at or near a proximal portion of stent 1220, and distal pressure sensor 1240 is disposed at or near a distal portion of stent 1220. The catheter branch, or a portion thereof, can be placed within a vessel or lumen such as a renal artery 1250 having a stenosis or lesion 1260. The catheter system can be used to assess the degree of stenosis within renal artery 1250. For example, catheter branch 1210 may be advanced into or toward renal artery 1250 such that the undeployed stent 1260 is disposed within or at, or otherwise near, target lesion 1260. A differential pressure across at least a portion of the lesion can be determined based on pressure sensor measurements taken from proximal pressure sensor 1230 and distal pressure sensor 1240. If a differential pressure across the lesion or a portion thereof is determined to be sufficiently significant to warrant intervention, the stent may be deployed. For example, a differential pressure value may include a mean pressure value of 120 mmHg at the proximal face of the lesion and 80 mmHg distally. As shown in FIG. 12B, stent 1220 can be deployed to an expanded configuration, so as to open or apply an expansive force on lesion 1260. As shown here, lesion or stenosis 1260 is opened or otherwise reduced. In some embodiments, pressure measurements before, during, or after stenting are taken from the branch sensors located proximal and distal to the stenosis. Such stenting measurements allow for evaluation of the effectiveness of the stenting procedure. In some embodiments according to the present invention, a flow sensor 1270 may be embedded within or coupled with catheter branch 1210. Measurements from flow sensor 1270 can be used to help assess any relative increase in renal artery blood flow after or during stent placement, for example by making measurements before, during, or after stent placement. The catheter system can be used to perform a navigation procedure to place the stent at a desired location relative to the lesion. This can be accomplished in a case where the catheter branch carries a thru-lumen, where a port is at the tip of the branch, and a stent delivery system can be delivered through the bifurcated infusion catheter, as to exit from the distal port of the catheter branch. Hence, a bifurcated renal system can be employed as a sensing device, as well as a treatment device, as part of a stenosis or lesion intervention treatment within a renal artery. In some embodiments, renal catheter branch 1210 may also include a dilation balloon, in addition to or instead of a stent, and the dilation balloon can be used to treat a renal artery stenosis.

A bifurcated renal catheter system having one or more pressure sensing elements, and optionally one or more flow rate sensors, may be also be used for techniques that involve laser phototherapy for the treatment of renal artery stenosis. As such, the catheter system may be used to evaluate the degree of a stenosis, as a determination factor for optimal or desired treatment. In some embodiments, a renal catheter system may also house a laser emitter on one or more catheter branches. Such an integrated system can allow for both the assessment and treatment of a renal artery stenosis. The pressure and flow sensing elements of the catheter may be used to assess the relative effectiveness in treating the stenosis acutely after or during phototherapy. Many of the renal catheter systems and methods described herein are well suited for use in the analysis or treatment, or both, of a renal artery stenosis. A pressure sensor may be embedded into or coupled with a section of catheter shaft exposed from the distal tip of the guide sheath, as shown in FIG. 10. Such a pressure sensor at this location can be used to identify the effectiveness of a renal artery stenosis treatment, for example by determining to what extent previous hypertensive systemic blood pressure is restored to normal levels.

F. Analysis of Clot/Particle Entry into the Renal Arteries

As certain interventions and drug therapies may promote clot or debris formation and migration into the kidneys via the renal arteries, for example as created during renal stenting procedures, a predictive marker for potentially reduced renal function may be debris or particle concentration within renal blood flow. Embodiments of the present invention may be used to assess such a marker in a patient. For example, a bifurcated renal catheter system may include a sensor, embedded with or coupled with a catheter branch, which is capable of detecting the concentration of particles within the renal artery blood stream, or counting individual particulates within the renal artery blood stream, or both. During or as part of a targeted renal therapy treatment or other intervention, such a particulate or clot measurement within the renal artery blood flow can be used as a basis for making a determination whether to administer vasodilative or clot dissolving agents, or both, to the patient undergoing the procedure, in order to minimize or reduce any potentially detrimental effects of stray embolic material on renal function.

2. Module Systems

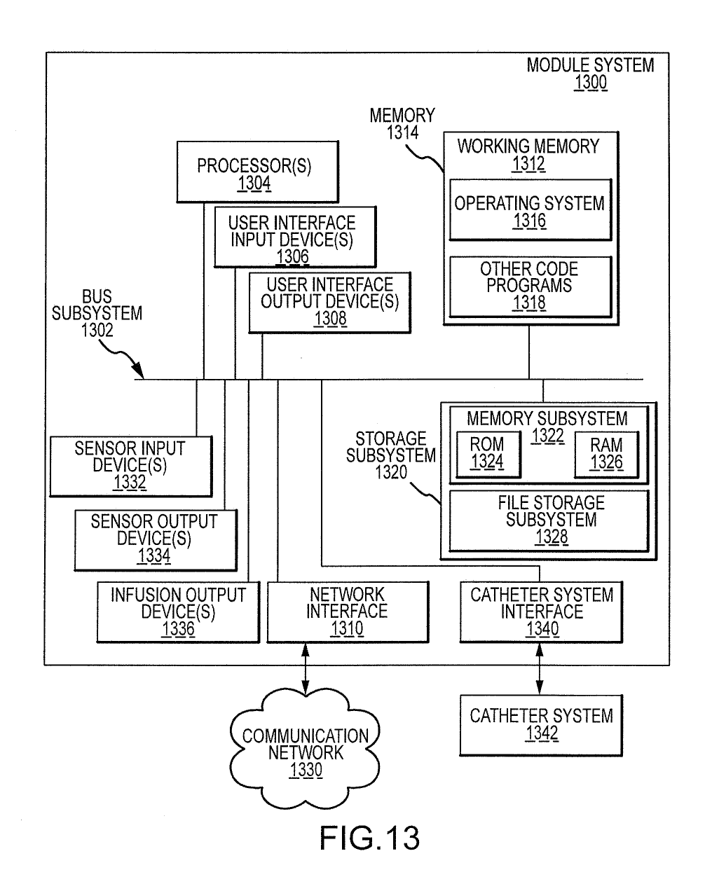

FIG. 13 is a simplified block diagram of an exemplary module system that broadly illustrates how individual system elements for a module system 1300 may be implemented in a separated or more integrated manner. Module system 1300 is well suited for monitoring physiological parameters in a patient and for controlling pharmacological interventions administered to the patient. Module system 1300 is shown comprised of hardware elements that are electrically coupled via a bus subsystem 1302, including one or more processors 1304, one or more input devices 1306 such as user interface input devices, one or more output devices 1308 such as user interface output devices, a network interface 1310, and a catheter system interface 1340 that can receive signals from and transmit signals to catheter system 1342.

In some embodiments module system 1300 also comprises software elements, shown as being currently located within working memory 1312 of memory 1314, including an operating system 1316 and other code 1318, such as a program designed to implement methods of the invention.

Likewise, in some embodiments module system 1300 may also include a storage subsystem 1320 that can store the basic programming and data constructs that provide the functionality of the various embodiments of the present invention. For example, software modules implementing the functionality of the methods of the present invention, as described herein, may be stored in storage subsystem 1320. These software modules are generally executed by the one or more processors 1304. In a distributed environment, the software modules may be stored on a plurality of computer systems and executed by processors of the plurality of computer systems. Storage subsystem 1320 can include memory subsystem 1322 and file storage subsystem 1328. Memory subsystem 1322 may include a number of memories including a main random access memory (RAM) 1326 for storage of instructions and data during program execution and a read only memory (ROM) 1324 in which fixed instructions are stored. File storage subsystem 1328 can provide persistent (non-volatile) storage for program and data files, and may include tangible storage media which may optionally embody patient, treatment, assessment, or other data. File storage subsystem 1328 may include a hard disk drive, a floppy disk drive along with associated removable media, a Compact Digital Read Only Memory (CD-ROM) drive, an optical drive, DVD, CD-R, CD-RW, solid-state removable memory, other removable media cartridges or disks, and the like. One or more of the drives may be located at remote locations on other connected computers at other sites coupled to module system 1300. The modules implementing the functionality of the present invention may be stored by file storage subsystem 1328. In some embodiments, the software or code will provide protocol to allow the module system 1300 to communicate with communication network 1330. Often such communications will include dial-up or internet connection communications.

It is appreciated that system 1300 can be configured to carry out various methods of the present invention. For example, processor component or module 1304 can be a microprocessor control module configured to receive physiological parameter signals from sensor input device or module 1332 or user interface input device or module 1306, and to transmit treatment signals to infusion output device or module 1336, user interface output device or module 1308, network interface device or module 1310, or any combination thereof. Each of the devices or modules according to embodiments of the present invention can include one or more software modules on a computer readable medium that is processed by a processor, or hardware modules, or any combination thereof. Any of a variety of commonly used platforms, such as Windows, MacIntosh, and Unix, along with any of a variety of commonly used programming languages, may be used to implement embodiments of the present invention.

User interface input devices 1306 may include, for example, a touchpad, a keyboard, pointing devices such as a mouse, a trackball, a graphics tablet, a scanner, a joystick, a touchscreen incorporated into a display, audio input devices such as voice recognition systems, microphones, and other types of input devices. User input devices 1306 may also download a computer executable code from a tangible storage media or from communication network 1330, the code embodying any of the methods of the present invention. It will be appreciated that terminal software may be updated from time to time and downloaded to the terminal as appropriate. In general, use of the term “input device” is intended to include a variety of conventional and proprietary devices and ways to input information into module system 1300.

User interface output devices 1306 may include, for example, a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide a non-visual display such as via audio output devices. In general, use of the term “output device” is intended to include a variety of conventional and proprietary devices and ways to output information from module system 1300 to a user.

Bus subsystem 1302 provides a mechanism for letting the various components and subsystems of module system 1300 communicate with each other as intended. The various subsystems and components of module system 1300 need not be at the same physical location but may be distributed at various locations within a distributed network. Although bus subsystem 1302 is shown schematically as a single bus, alternate embodiments of the bus subsystem may utilize multiple busses.

Network interface 1310 can provide an interface to an outside network 1330 or other devices. Outside communication network 1330 can be configured to effect communications as needed or desired with other parties. It can thus receive an electronic packet from module system 1300 and transmit any information as needed or desired back to module system 1300. In addition to providing such infrastructure communications links internal to the system, the communications network system 1330 may also provide a connection to other networks such as the internet and may comprise a wired, wireless, modem, and/or other type of interfacing connection.

It will be apparent to the skilled artisan that substantial variations may be used in accordance with specific requirements. For example, customized hardware might also be used and/or particular elements might be implemented in hardware, software (including portable software, such as applets), or both. Further, connection to other computing devices such as network input/output devices may be employed. Module terminal system 1300 itself can be of varying types including a computer terminal, a personal computer, a portable computer, a workstation, a network computer, or any other data processing system. Due to the ever-changing nature of computers and networks, the description of module system 1300 depicted in FIG. 13 is intended only as a specific example for purposes of illustrating one or more embodiments of the present invention. Many other configurations of module system 1300 are possible having more or less components than the module system depicted in FIG. 13. Any of the modules or components of module system 1300, or any combinations of such modules or components, can be coupled with, or integrated into, or otherwise configured to be in connectivity with, any of the catheter system embodiments disclosed herein. Relatedly, any of the hardware and software components discussed above can be integrated with or configured to interface with other medical assessment or treatment systems used at other locations.

In some embodiments, the module system 1300 can be configured to receive a physiological parameter of a first renal artery, and optionally receive a physiological parameter of a second renal artery, at an input module. Physiological parameter data can be transmitted to an assessment module where a physiological profile is determined. The profile can be output to a system user via an output module. In some cases, the module system 1300 can determine a treatment protocol for the patient, based on a physiological parameter or profile, for example by using a treatment module. The treatment can be output to a system user via an output module. Optionally, certain aspects of the treatment can be determined by an infusion output device, and transmitted to a catheter system or an infusion pump of a catheter system. Any of a variety of data related to the patient can be input into the module system, including age, weight, sex, treatment history, medical history, and the like. Parameters of treatment regimens or diagnostic evaluations can be determined based on such data.

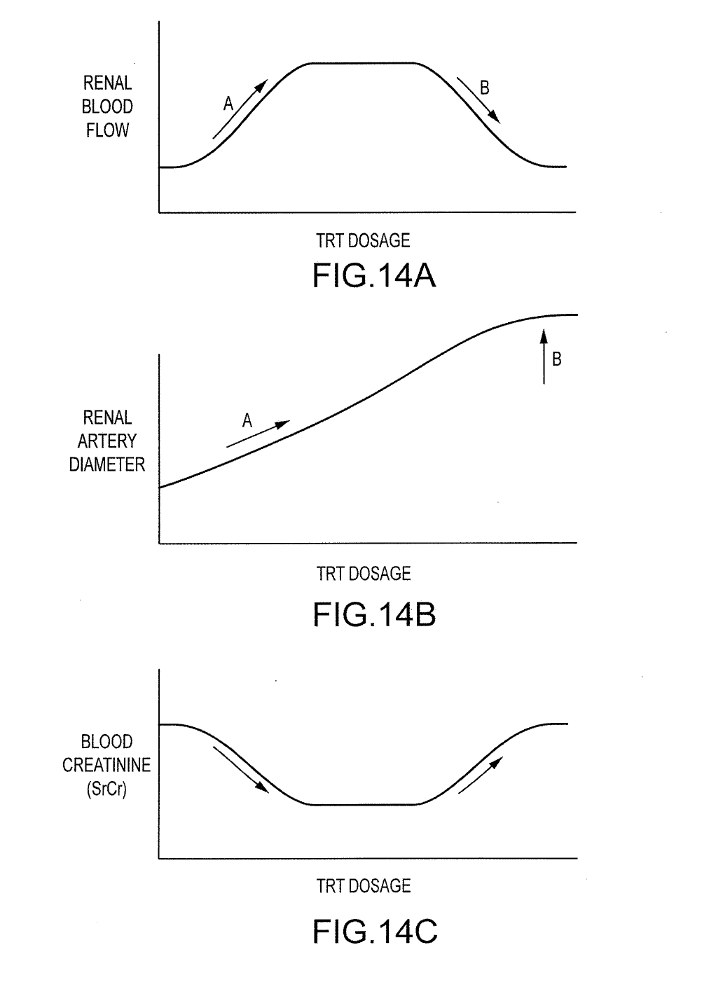

FIGS. 14A to 14C schematically illustrate plots of a targeted renal therapy (TRT) dosage versus certain physiological parameters associated with renal function, according to embodiments of the present invention. Hence, these figures depict dosing effects on physiological parameters, which can be used as a basis for determining an assessment of a physiological parameter of a patient, or for determining a pharmacological regimen for a patient. FIG. 14A shows a graph of renal blood flow as a function of targeted renal therapy dosage. As indicated by arrow A, an increase in blood flow may be due to focalized effects of a pharmacological agent on the kidney. A majority or substantial portion of the agent may be excreted by the kidney, and not reintroduced into the systemic circulation. As indicated by arrow B, a decrease in blood flow may be due to an excess of pharmacological agent. The kidney may not be able to properly or sufficiently excrete the agent, and hence the treatment presents a higher systemic exposure. FIG. 14B shows a graph of renal artery diameter as a function of targeted renal therapy dosage. As indicated by arrow A, an increase in artery diameter may be due to vasodilative effects of the administered pharmacological agent. As indicated by arrow B, the patient may experience a drug saturation point, where biomechanical mechanisms for vasodilation reach or approach capacity. FIG. 14C shows a graph of renal or systemic blood creatinine (SrCr) as a function of targeted renal therapy dosage. As indicated by arrow A, a decrease in blood creatinine may be due to local delivery of a pharmacological agent to the kidney and excretion of most or a substantial portion of the administered agent. Thus, improved kidney function may result. As indicated by arrow B, an increase in blood creatinine may be due to excess agent. The kidney may be unable to properly or sufficiently excrete the agent, leading to a higher systemic exposure for the patient. Thus, a decline in kidney function may result.

Module systems and methods, and sensing and delivery configurations and techniques disclosed herein are well suited for use in a variety of local delivery catheters, including without limitation those described in U.S. patent application Ser. No. 11/084,738 filed Mar. 16, 2005; U.S. patent application Ser. No. 11/295,735 filed Dec. 5, 2005; U.S. Pat. No. 7,104,981 issued Sep. 12, 2006; U.S. patent application Ser. No. 11/084,434 filed Mar. 18, 2005; U.S. patent application Ser. No. 11/303,554 filed Dec. 16, 2005; U.S. patent application Ser. No. 11/073,421 filed Mar. 4, 2005; U.S. patent application Ser. No. 11/129,101 filed May 13, 2005; U.S. patent application Ser. No. 11/233,562 filed Sep. 22, 2005; U.S. patent application Ser. No. 11/347,008 filed Feb. 3, 2006; U.S. patent application Ser. No. 11/167,056 filed Jun. 23, 2005; U.S. patent application Ser. No. 11/758,417 filed Jun. 5, 2007; U.S. patent application Ser. No. 11/241,749 filed Sep. 29, 2005; and U.S. patent application Ser. No. 11/548,565 filed Oct. 11, 2006. The content of each of these filings in incorporated herein by reference.

While the above provides a full and complete disclosure of certain embodiments of the present invention, various modifications, alternate constructions and equivalents may be employed as desired. Therefore, the above description and illustrations should not be construed as limiting the invention, which is defined by the appended claims.

What is claimed is:

1. A method of assessing a physiological profile of a patient, comprising:
advancing a catheter shaft of a bifurcated renal catheter system into an aorta of the patient;
deploying a first catheter branch of the bifurcated renal catheter system into a first renal artery of the patient, and a second catheter branch of the bifurcated renal catheter system into a second renal artery of the patient;
detecting a physiological parameter of the first renal artery, and optionally detecting a physiological parameter of the second renal artery, with a sensing mechanism of the bifurcated renal catheter system; and
assessing the physiological profile of the patient based on the physiological parameter of the first renal artery, on the physiological parameter of the second renal artery, or on the physiological parameter of the first renal artery and the physiological parameter of the second renal artery.

2. The method according to claim 1, wherein the first catheter branch comprises a first branch sensing element, and the second catheter branch comprises a second branch sensing element, the method further comprising detecting the physiological parameter of the first renal artery with the first branch sensing element, and optionally detecting the physiological parameter of the second renal artery with the second branch sensing element.

3. The method according to claim 1, further comprising:
advancing a catheter shaft of a second bifurcated renal catheter system into an inferior vena cava of the patient;
deploying a first catheter branch of the second bifurcated renal catheter system into a first renal vein of the patient, and a second catheter branch of the second bifurcated renal catheter system into a second renal vein of the patient;
detecting a physiological parameter of the first renal vein, and optionally detecting a physiological parameter of the second renal vein, with a sensing mechanism of the second bifurcated renal catheter system; and
assessing the physiological profile of the patient based on the physiological parameter of the first renal vein, on the physiological parameter of the second renal vein, or on the physiological parameter of the first renal vein and the physiological parameter of the second renal vein.

4. The method according to claim 1, further comprising:
delivering a first amount of a first pharmacological agent to the first renal artery, and optionally delivering a second amount of a second pharmacological agent to the second renal artery, with an agent delivery mechanism of the bifurcated renal catheter system;
detecting a subsequent physiological parameter of the first renal artery, and optionally detecting a subsequent physiological parameter of the second renal artery, with the sensing mechanism of the bifurcated renal catheter system; and
assessing an effect of the first amount of the first pharmacological agent on the physiological profile of the patient based on the subsequent physiological parameter of the first renal artery, and optionally assessing an effect of the of the second amount of the second pharmacological agent on the physiological profile of the patient based on the subsequent physiological parameter of the second renal artery.

5. The method according to claim 6, wherein the first pharmacological agent and the second pharmacological agent each comprise a member selected from the group consisting of a contrast solution, a chemotherapy agent, an antioxidant, sodium bicarbonate, acetylcysteine, a chelation agent, an anti-inflammatory agent, fenoldopam mesylate, a vasodilator, prostaglandin, a diuretic, a loop diuretic, furosemide, an antibiotic agent, a bactericidal agent, a bacteriostatic agent, a neurohormonally active agent, a natriuretic peptide, A-type natriuretic peptide, B-type natriuretic peptide, C-type natriuretic peptide, a synthetic natriuretic peptide, and a bio-engineered natriuretic peptide.

6. The method according to claim 1, further comprising:
performing a surgical procedure on the patient;
detecting a subsequent physiological parameter of the first renal artery, and optionally detecting a subsequent physiological parameter of the second renal artery, with the sensing mechanism of the bifurcated renal catheter system; and
assessing an effect of the surgical procedure on the physiological profile of the patient based on the subsequent physiological parameter of the first renal artery, and optionally assessing the effect of the surgical procedure on the physiological profile of the patient based on the subsequent physiological parameter of the second renal artery.

7. The method according to claim 6, wherein the surgical procedure comprises a member selected from the group consisting of a stenting procedure, a bypass procedure, an angiographic procedure, a percutaneous coronary intervention, and an invasive surgical procedure.

8. The method according to claim 1, wherein the physiological parameter of the first or the second renal artery comprises a blood concentration of a physiological marker selected from the group consisting of aldosterone, renin, angiotensin II, serum creatinine (SrCr), urea, neutrophil gelatinase-associated lipocalin (NGAL), cystanin C, acetylcholine, bradykinin, blood urea nitrogen (BUN), calcium, potassium, sodium, chloride, bicarbonate, oxygen, nitric oxide (NO), nitric oxide synthase (NOS), reactive oxygen species (ROS), iron, an iron-based biochemical derivative, and a blood pH.

9. The method according to claim 1, wherein the physiological parameter of the first or the second renal artery comprises a blood concentration of an inflammatory marker selected from the group consisting of a polymorphonuclear leukocyte (PMN), an interleukin-8 (IL-8), IL-13, and IL-17.

10. The method according to claim 1, wherein the physiological parameter of the first or the second renal artery comprises a blood chemotaxis indicator selected from the group consisting of a chemotaxis protein (MCP), methylesterase, and methyltransferase.

11. The method according to claim 1, wherein the physiological parameter of the first or the second renal artery comprises a blood concentration of a contrast solution.

12. The method according to claim 1, wherein the physiological parameter of the first or the second renal artery comprises a physical marker selected from the group consisting of a renal artery blood flow velocity, a volumetric blood flow rate, a total renal blood flow, an inner arterial wall shear stress, a pressure, a luminal diameter, a stenosis measure, a clot measure, a particle measure, and a temperature.

13. The method according to claim 1, wherein the sensing mechanism comprises a member selected from the group consisting of an ultrasonic transducer sensor, an expandable and retractable frame, a flow guided sensor, a balloon, a mesh umbrella, a flow meter, a shear stress sensor, a pressure sensor, a temperature sensor, a flow velocity sensor, a volumetric flow sensor, a Doppler sensor, and a biochemical sensor.

14. A bifurcated renal catheter system for assessing a physiological profile of a patient, comprising:
a catheter having a shaft coupled with a first catheter branch and a second catheter branch; and
a sensing mechanism having a first sensor coupled with the first catheter branch, and optionally a second sensor coupled with the second catheter branch.

15. The system according to claim 14, wherein the sensing mechanism comprises a member selected from the group consisting of an ultrasonic transducer sensor, an expandable and retractable frame, a flow guided sensor, a balloon, a multi-prong balloon, a mesh umbrella, a flow meter, a shear stress sensor, a pressure sensor, a temperature sensor, a flow velocity sensor, a volumetric flow sensor, a Doppler sensor, a flow rate sensor, a force transducer, a stent, and a biochemical sensor.

16. The system according to claim 14, further comprising a monitoring system coupled with the sensing mechanism.

17. The system according to claim 14, wherein the first catheter branch comprises a first infusion port, and the second catheter branch comprises a second infusion port.

18. The system according to claim 17, further comprising a guide sheath configured to receive the catheter shaft, a system monitor coupled with the sensing mechanism, and an infusion pump coupled with the first and second infusion ports.

19. A method of determining a physiological profile of a patient, comprising:
receiving a physiological parameter of a first renal artery, and optionally receiving a physiological parameter of the second renal artery, at an input module of a monitor and control system, the input module comprising a tangible medium embodying machine-readable code; and
determining the physiological profile of the patient with an assessment module of the monitor and control system, the assessment module comprising a tangible medium embodying machine-readable code.

20. The method according to claim 19, further comprising determining a patient treatment, based on the physiological profile, with a treatment module of the monitor and control system, the treatment module comprising a tangible medium embodying machine-readable code.

21. The method according to claim 20, wherein determining the patient treatment comprises determining a treatment agent and calculating an amount of the treatment agent to be delivered to the first renal artery of the patient.

22. The method according to claim 19, further comprising:
advancing a catheter shaft of a bifurcated renal catheter system into an aorta of the patient;
deploying a first catheter branch of the bifurcated renal catheter system into the first renal artery of the patient, and deploying a second catheter branch of the bifurcated renal catheter system into the second renal artery of the patient;
detecting the physiological parameter of the first renal artery, and optionally detecting the physiological parameter of the second renal artery, with a sensing mechanism of the bifurcated renal catheter system.

23. The method according to claim 19, further comprising administering a treatment to the patient, and determining a subsequent physiological profile of the patient after or while administering the treatment the patient.

24. A bifurcated renal catheter system for assessing a physiological profile of a patient, comprising:
a catheter having a shaft coupled with a first catheter branch and a second catheter branch;
a sensing mechanism having a first sensor coupled with the first catheter branch, and optionally a second sensor coupled with the second catheter branch; and
a monitor and control system comprising an input module having a tangible medium embodying machine-readable code configured to receive an input from the sensing mechanism, and an assessment module having a tangible medium embodying machine-readable code configured to assess the physiological profile of the patient based on the input.

25. A module system for determining a treatment for a patient, comprising:
a catheter having a shaft coupled with a first catheter branch and a second catheter branch;
a sensing mechanism having a first sensor coupled with the first catheter branch, and optionally a second sensor coupled with the second catheter branch; and
a monitor and control system comprising an input module having a tangible medium embodying machine-readable code configured to receive an input from the sensing mechanism, an assessment module having a tangible medium embodying machine-readable code configured to perform an assessment of the physiological profile of the patient based on the input, and a treatment module having a tangible medium embodying machine-readable code configured to determine a patient treatment based on the assessment.
